(19) United States
(12) Patent Application Publication
(10) Pub. No.: US 2003/0064001 A1
(43) Pub. Date: Apr. 3, 2003

(54) System for the decontamination of fluid products using light

(76) Inventors: William M. Fries, San Diego, CA (US); Eddie Lee Brown, San Diego, CA (US); Benjamin H. Holko, San Diego, CA (US); Kenton J. Salisbury, San Diego, CA (US); Richard E. May, San Diego, CA (US); John S. Thompson, San Diego, CA (US); Jeffrey M. Holloway, San Diego, CA (US)

Correspondence Address: FITCH EVEN TABIN AND FLANNERY, 120 SOUTH LA SALLE STREET, SUITE 1600, CHICAGO, IL, 60603-3406, US

(21) Appl. No.: 09/976,597

(22) Filed: Oct. 12, 2001

Related U.S. Application Data
(60) Provisional application No. 60/291,850, filed on May 17, 2001.

(51) Int. Cl. A61L 2/00; B01J 19/08

(52) U.S. Cl. 422/22; 422/24; 422/186.3; 250/455.11

(57) ABSTRACT

A fluid treatment system consists of a light source for providing light and a flexible treatment chamber having an input port and an output port, at least a portion of the flexible treatment chamber positioned to receive the light. The at least the portion of the flexible treatment chamber is transmissive to at least 1% of the light having at least one wavelength within a range of 170 to 2600 nm and the flexible treatment chamber is adapted to allow a fluid to be treated to be flowed via the input port therethrough at a specified rate and out the output port. The light source illuminates the fluid as it flows through the flexible treatment chamber in order to deactivate pathogens within the fluid. In some embodiments, the light source is a pulsed light source for emitting short duration, pulses of polychromatic light.

This application is a Non-Provisional application of and claims priority under 35 U.S.C. 119(e) to U.S. Provisional Application No. 60/291,850, of Fries et al., entitled SYSTEM FOR DECONTAMINATION OF FLUID PRODUCTS USING BROAD SPECTRUM LIGHT, filed May 17, 2001, which is incorporated herein in its entirety by reference.

This patent document relates to the following patent document filed concurrently herewith, which is incorporated herein in its entirety by reference: U.S. Patent Application No. ______, of Fries et al.; entitled FLUID FLOW PATH FOR A FLUID TREATMENT SYSTEM USING LIGHT FOR THE DECONTAMINATION OF FLUID PRODUCTS, Docket No. 71715; now U.S. Patent No. ______.

### BACKGROUND OF THE INVENTION

1. Field of the Invention

The present invention relates generally to fluid treatment systems, and more specifically to fluid treatment systems using a light source for treating fluid products. Even more specifically, the present invention relates to fluid treatment systems using light, and methods of use, for deactivating pathogens in fluid products, such as biological fluids including blood products.

2. Discussion of the Related Art

Many techniques exist to purify or deactivate unwanted pathogens contained within fluid products. Such pathogens may include microorganisms, viruses, bacteria, fungus or other harmful substances. Some known fluid treatments include heat treatment, chemical treatment, and light treatment (e.g., exposing the fluid product to continuous wave ultraviolet (UV) light).

Most fluid treatment systems are designed to allow the fluid product to be treated to flow through a treatment zone for exposure to the specific treatment. For example, in a light-based fluid treatment system, a conduit or other fluid flow path structure carries the fluid product near a light source that illuminates the fluid as is flows thereby. Such systems are often designed to accommodate a variety of fluid products, such as fluids for pharmaceutical or medical use. Often, when treating different fluid products, such fluid treatment systems must be sterilized in between different uses such that fluid products from previous runs do not contaminate current fluid products to be treated. This can be a difficult task depending on the physical complexity and structure of the flow path structure of the fluid within the treatment system.

Additionally, special care must be taken when the fluid product to be treated is a biological fluid, such as a blood product, so as to avoid damaging the biological fluid (e.g., reducing the protein activity of the blood product) while at same time deactivating pathogens or other contaminants. It is often important not to damage these biological fluid products since the product may be unusable if damaged too much. Additionally, certain biological fluids can be very expensive and not easily replaceable.

### SUMMARY OF THE INVENTION

The present invention advantageously addresses the needs above as well as other needs by providing a unique fluid treatment system, components and methods of use for the deactivation of pathogens in fluids, such as biological fluids.

In one embodiment, the invention can be characterized as a fluid treatment system including a light source for providing light and a flexible treatment chamber having an input port and an output port, at least a portion of the flexible treatment chamber positioned to receive the light. The at least the portion of the flexible treatment chamber is transmissive to at least 1% of the light having at least one wavelength within a range of 170 to 2600 nm and the flexible treatment chamber is adapted to allow a fluid to be treated to be flowed via the input port therethrough at a specified rate and out the output port. The light source illuminates the fluid as it flows through the flexible treatment chamber in order to deactivate pathogens within the fluid.

In another embodiment, the invention can be characterized as a light treatment system for treating fluid products including a light source for providing light, a treatment chamber positioned to receive the light and for allowing the fluid products to be flowed therethrough and a support structure supporting the treatment chamber and defining at least one dimensional boundary of a treatment zone of the treatment chamber. At least a portion of the treatment chamber and at least a portion of the support structure is transmissive to at least 1% of the light having at least one wavelength within a range of 170 to 2600 nm. The light source illuminates the fluid products as they flow through the treatment chamber in order to deactivate pathogens within the fluid products.

In a further embodiment, the invention may be characterized as a disposable light treatment chamber including a flexible flow chamber transmissive to at least 1% of a light treatment having at least one wavelength within a range of 170 to 2600 nm, the flexible flow chamber adapted to allow a fluid to be flowed therethrough and illuminated with the light treatment such that pathogens within the fluid are deactivated by the light treatment. Also included is an input port formed at one part of the flexible flow chamber and adapted to receive a flow of the fluid to be treated and an output port formed at another part of the flexible flow chamber adapted to receive the flow of the fluid having been treated with the light treatment.

In yet another embodiment, the invention may be characterized as a light treatment device to be illuminated with light for deactivating pathogens within fluid products including a cartridge body comprising a first part and a second part, a first light transmissive window of the first part and a flexible treatment chamber positioned against the first light transmissive window. At least a portion of the flexible treatment chamber is transmissive to at least 1% of the light having at least one wavelength within a range of 170 to 2600 nm. The fluid to be treated with the light flows through the flexible treatment chamber. Also included is a plate portion of the second part, the plate portion positioned against the flexible treatment chamber, wherein the plate portion restrains the flexible treatment chamber against the first light transmissive window in order to define at least one dimensional boundary of a fluid flow path for the fluid within the flexible treatment chamber.

In yet another embodiment, the invention may be characterized as a method of fluid decontamination including the steps of: flowing a fluid product through a flexible treatment chamber, the flexible treatment chamber being light transmissive to at least 1% of a light treatment having at least one wavelength within a range of 170 to 2600 nm; illuminating the fluid product with the light as the fluid product is flowed through the flexible treatment chamber; and deactivating pathogens within the fluid product.

### BRIEF DESCRIPTION OF THE DRAWINGS

The above and other aspects, features and advantages of the present invention will be more apparent from the following more particular description thereof, presented in conjunction with the following drawings wherein:

FIGS. 1, 2 and 3 are a front perspective view, a rear perspective view and a front view, respectively, of a fluid treatment system using a light source emitting e.g., pulsed polychromatic light, such as broad spectrum pulsed light (BSPL), according to one embodiment of the invention;

FIG. 4 is an external view of the fluid treatment system of FIGS. 1-3.;

FIG. 5 is a perspective view of a syringe mount assembly of the fluid treatment system of FIGS. 1-3 according to one embodiment of the invention;

FIG. 6 is a schematic view of the fluid flow path components of the fluid treatment system of FIGS. 1-3 according to another embodiment of the invention;

FIGS. 7A and 7B, are a perspective view and a side view, respectively, of one embodiment of a treatment chamber of the fluid flow path of FIG. 6;

FIG. 7C is a schematic view of a transition from a circular flow profile to a substantially flat profile at the input and output of the treatment chamber of FIGS. 7A and 7B according to another embodiment of the invention;

FIG. 8 is an exploded view of one embodiment of a cartridge as shown in FIGS. 1-3 illustrating the treatment chamber of FIG. 7 positioned therein;

FIGS. 9A and 9B are cross sectional views of the cartridge of FIG. 8 containing the treatment chamber of FIGS. 7A-7B according to one embodiment of the invention;

FIG. 10 is a perspective view of the cartridge of FIG. 8 as positioned within the cartridge registration plate of the fluid treatment system of FIGS. 1-3 in accordance with one embodiment of the invention;

FIG. 11 is a perspective view of another embodiment of the fluid treatment system of FIGS. 1-3;

FIG. 12 is a perspective view of a flat, disposable treatment chamber that may be used in the fluid treatment system of FIGS. 1-3 in accordance with another embodiment of the invention;

FIG. 13 is a perspective view of a reusable, non-disposable treatment chamber according to another embodiment of the invention;

FIG. 14 is a perspective view of a treatment chamber that may be used in the fluid treatment system of FIGS. 1-3 in accordance with another embodiment of the invention;

FIGS. 15A and 15B are a simplified front view and side view, respectively, illustrating the relationship between the treatment chamber, the light source and the respective process monitors according to one embodiment of the invention;

FIG. 16 is a simplified side view of a variation of the process monitoring system of FIG. 15B according to another embodiment of the invention;

FIG. 17A is a simplified perspective view of a detector array that is used to obtain the spectral profile of the light treatment across the entire treatment chamber according to yet another embodiment of the invention;

FIG. 17B is a simplified perspective view of process monitors integrated on an adjustable x-y translation table used to obtain the spectral profile of the light treatment across different portions of the treatment chamber according to yet another embodiment of the invention;

FIG. 18 is a simplified side view of a treatment chamber including a spectral filter positioned between the treatment chamber and the light source according to another embodiment of the invention;

FIG. 19 is a simplified side view of a treatment chamber including a device to cool the treatment chamber due to the heat energy of the light illuminating the treatment chamber according to another embodiment of the invention;

FIG. 20A is a system level diagram is shown for a fluid treatment system according to one embodiment of the invention;

FIG. 20B is a simplified schematic drawing of production fluid treatment system scaled to continuously treat fluids according to one embodiment of the invention;

FIG. 21 is a graph plotting the percentage of protein activity remaining vs. the number of flashes used in EXAMPLE 1;

FIG. 22 is a graph plotting the log reduction of E. coli within a test fluid vs the number of flashes at a high and at a low fluence level according to EXAMPLE 2;

FIG. 23 is a graph plotting the log reduction of E. coli within a test fluid vs time in an extended run test according to EXAMPLE 3;

FIG. 24 is a graph plotting the radiant energy across a wavelength spectrum of light treatment transmitting through a treatment chamber according to EXAMPLE 7; and

FIGS. 25 and 26 are graphs plotting the percentage of protein recovery or protein activity vs. the total energy of BSPL for various fluence levels/flash for Beta-galactosidase in water and BSA, respectively.

Corresponding reference characters indicate corresponding components throughout the several views of the drawings.

### DETAILED DESCRIPTION OF THE INVENTION

The following description is not to be taken in a limiting sense, but is made merely for the purpose of describing the general principles of the invention. The scope of the invention should be determined with reference to the claims.

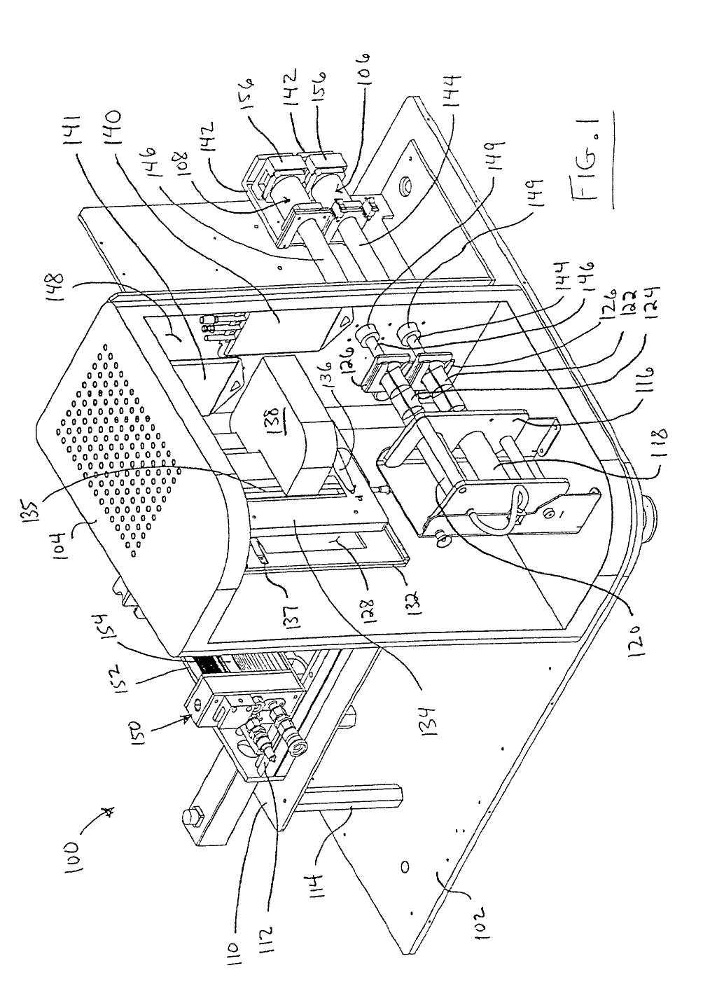

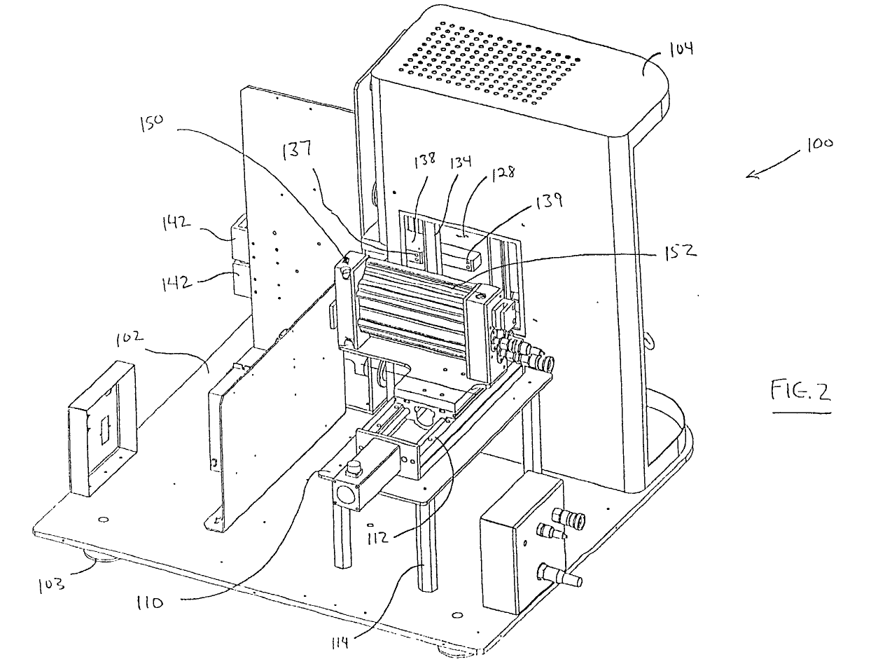

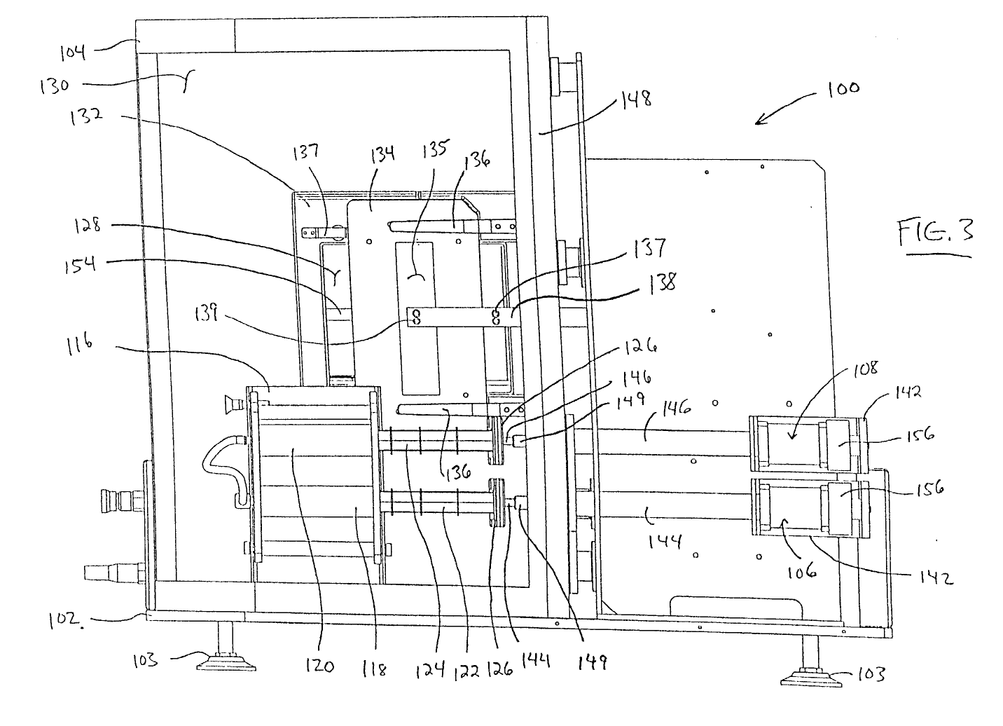

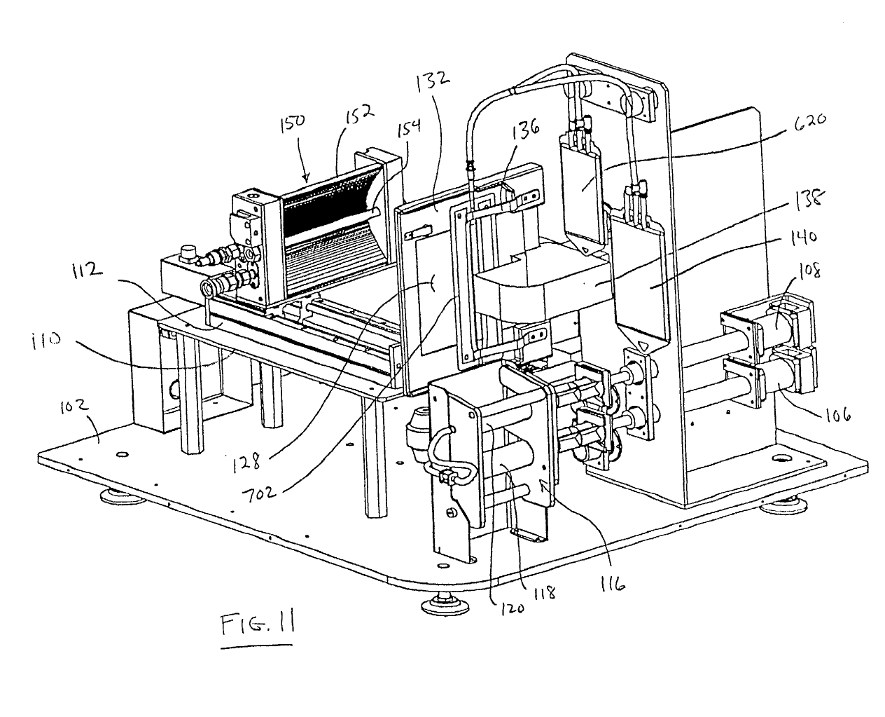

Referring first to FIGS. 1-3, several views are shown a fluid treatment system that uses a light source that emits pulsed polychromatic light, for example, such as broad spectrum pulsed light (BSPL), according to one embodiment of the invention. FIG. 1 is a front perspective view, FIG. 2 is a rear perspective view, and FIG. 3 is front view of the fluid treatment system. Illustrated is the fluid treatment system 100 including a base plate 102, support levelers 103, a treatment area enclosure 104, actuator assemblies 106 and 108 (also referred to generically as pumps), a lamp support plate 110, a linear slide servo drive 112 and support posts 114. The actuator assemblies 106 and 108 are held in place by actuator assembly brackets 142 and each includes linear actuators 144 and 146 that extend through wall 148 at seals 149. At the end of the linear actuators 144 and 146 are respective brackets 126. The lamp support plate 110 holds a lamp assembly 150 including a reflector 152 and a light source 154 within profile of the reflector 152. It is noted that in preferred embodiments, the light source 154 is a pulsed light source, such as a flashlamp; however, in other embodiments, the light source 154 is a continuous wave light source (e.g., a UV lamp) or other pulsed light source operating at a single wavelength or operating within a range of wavelengths. It is noted that the light source 154 is partially viewable through window 128 in FIG. 3 and is also illustrated in FIG. 10. The treatment area enclosure 104 houses a treatment area from the rest of interior of the fluid treatment system 100. The treatment area enclosure 104 includes a syringe mount mechanism 116 that holds syringes 118 and 120 (also referred to generically as fluid containers 118 and 120) including syringe plungers 122 and 124. The syringe plungers 122 and 124 are adapted to be held by the brackets 126. A cartridge registration plate 132 is positioned within wall 130 of the treatment enclosure 104. A window 128 is formed within the cartridge registration plate 132. The cartridge registration plate 132 is adapted to positionally align and hold a cartridge 134 that in some embodiments, contains a treatment chamber. The cartridge 134 is held in place by cartridge lock clips 136 and a cartridge retaining clip 137. A process monitor housing 138 is positioned in front of a cartridge window 135 of the cartridge 134. The process monitor housing 138 includes process monitors 137 and 139 facing toward the cartridge 132. Note that the process monitors 137 and 139 are seen through the window 128 in FIG. 2 while the positioning of the process monitors 137 and 139 is seen through the process monitor housing 138 in FIG. 3. Also included are an effluent bag 140 and a sample bag 141 (each of which may be generically referred to as a fluid collectors or fluid containers).

The fluid treatment system 100 is designed to treat fluids, including biological fluids, and their derivatives, e.g., blood, blood plasma, blood plasma derivatives, bioprocessing fluids and other fluid product, such as drugs and pharmaceuticals, especially bio-pharmaceuticals such as monoclonal antibodies, solutions such as a buffer, glucose and other sugar solutions, culture medias, as well as molecular biology and biochemistry reagents and other fluid product, etc., with light, for example, in this embodiment, with pulsed light. Generally, fluids are pumped from a fluid container (e.g., syringes 118 and 120), through a treatment chamber or treatment zone (such as formed within the cartridge 134) at a controlled rate while being illuminated with light from the light source 154, e.g., with pulses of light. The decontaminated fluid product continues to flow to the effluent bag 140, while samples are collected in the sample bag 141 for testing, evaluation and use. Advantageously, since in one embodiment, the light treatment is pulsed light, the entire fluid treatment process is designed to be complete within several seconds, e.g., less than 10 seconds; however, this depends upon the flow rate, size of the fluid containers, etc. The fluid treatment system 100 is designed to be adjustable and scalable, for example, to a continuous flow system and, in some embodiments, includes a disposable treatment chamber or treatment zone.

In order to pump the fluid to be treated through the treatment chamber at a desired rate, a pump mechanism is provided. In this embodiment, fluids that are to be treated with light are contained with syringe 118, while syringe 120 contains another fluid, such as water for injection. Alternatively, the syringe 120 may contain more of the fluid product to be treated. These syringes 118 and 120 are loaded into the syringe mount mechanism 116 such that the body of syringes 118 and 120 are within the syringe mount mechanism 116 and the syringe plungers 122 and 124 extend out of the syringe mount mechanism 116 such that the head of the syringe plungers are captured by brackets 126. Actuator assemblies 106 and 108 are mounted such that they float freely in the axis of the syringe plungers 122 and 124, but in this embodiment, are retained within the actuator assembly brackets 142 and within wall 148 of the treatment area enclosure 104. Linear actuators 144 and 146 (of actuator assemblies 106 and 108) extend linearly through wall 148 at seals 149 and are rigidly mounted to the brackets 126 holding the syringe plungers 122 and 124, respectively. In this embodiment, the actuator assemblies 106 and 108 each have about a 5-inch stroke.

The actuator assemblies 106 and 108 are designed to operate independently from each other or together depending on the parameters set by the operator. Upon activation of either or both of the actuator assemblies 106 and 108, the respective linear actuators 144 and 146 begin to move (extend) toward the syringe plungers 122 and 124. However, in this embodiment, since the actuator assemblies 106 and 108 float freely within the actuator assembly brackets 142, the entire actuator assemblies 106 and 108 each move slightly away from the syringe plungers 122 and 124 until it contacts a load cell contained within a load cell block 156 coupled to the actuator assembly brackets 142. Once the load cell is contacted, it signals to the system controller that a fluid flow is being established. Further motion of the actuator assemblies 106 and 108 away from the syringe plungers 122 and 124 is now prevented since the load cell blocks 156 are held in place by the actuator assembly brackets 142; thus, the linear actuators 144 and 146 apply a force against the syringe plungers 122 and 124, respectively, being retained by the brackets 126. The linear actuators 144 and 146 move independently, together, or consecutively at a constant rate set by the operator. The linear actuators 144 and 146 move the syringe plungers 122 and 124 into the syringes 118 and 120 forcing the fluid contained therein into tubing coupled to the cartridge 134. A flow rate is established by the linear velocity of the linear actuators 144 and 146. This rate is monitored by a linear encoder, e.g., a stepper drive, integrated into each of the linear actuators 144 and 146. It is noted in this embodiment, that the “pump mechanism” includes the syringe mount assembly 116, brackets 126, actuator assemblies 106 and 108, actuator assembly brackets 142, load cell blocks 156, seals 149 and the linear actuators 144 and 146. However, one skilled in the art will recognize that a number of different pumping mechanisms may be used to produce a flow of fluid through the cartridge 134 at a specified rate.

The fluids are forced to go through the tubing, which passes through a treatment chamber contained within the cartridge 134. For example, in this embodiment, the fluid is forced through the cartridge 134 from bottom to top. In other embodiments, the fluid flow may be from top to bottom or side-to-side or other arrangement depending upon the configuration of the system. In one embodiment, as the fluid passes through the cartridge 134, the lamp assembly 150 including the light source 154 emits light, e.g., short duration pulses of light, to decontaminate the fluid. The light deactivates pathogens contained within the fluid product. It is noted that in some embodiments, the light source 154 is a flashlamp; however, in other embodiments, the light source 154 may comprise a light source other than the flashlamp 154, such as a continuous light source or a pulsed laser light source. Thus, the lamp assembly 150 may emit pulsed light or continuous light energy depending on the specific system design. Additionally, the fluence level of the emitted light is carefully selected to minimize protein damage, in the event the fluid is a sensitive biological fluid, e.g., a blood plasma derivative or other bioprocessing media. The treated fluid continues to flow out of the cartridge 134 and is collected in the effluent bag 140. During the course of a fluid treatment run, a sample of the treated fluid is collected in the sample bag 141. In this embodiment, the fluid in the sample bag 141 is retained for its intended use, such as, testing evaluation, or use in application. Thus, the contents of the effluent bag are typically discarded.

In operation, the fluid within syringe 120, for example, water for injection (WFI) or other solution, such as saline, phosphate, etc., may be flowed prior to or at the same time as the fluid to be treated within syringe 118. Thus, the WFI may dilute the concentration of the fluid. Additionally, according to some embodiments, the WFI may be pumped through the cartridge 134 prior to pumping the fluid within syringe 118. As such, the WFI can be used to initialize the fluid treatment system and fill the fluid path to create back pressure and eliminate air bubbles, prior to flowing the actual fluids to be treated through. Furthermore, the WFI is used to verify the operating parameters of the emitted light as set by the operator. Once the light treatment is verified and the fluid treatment system is operating correctly, then actuator assembly 106 is operated and the fluid to be treated (e.g., in syringe 118) is flowed through the flow path. After the initialization and a steady flow of the fluid to be treated is flowing through the system, the sample is collected in the sample bag 141.

According to one embodiment, the lamp assembly 150 includes a light source 154 that provides pulsed polychromatic light, for example, broad spectrum pulsed light (BSPL), which illuminates and treats the fluid passing through the treatment chamber. BSPL is commonly produced by Xenon gas flashlamps, as known in the art. BSPL is pulsed light is in the form of high-intensity, short duration pulses of incoherent polychromatic light in a broad spectrum, also referred to as broad-spectrum pulsed light (i.e. BSPL) or broadband pulsed light. For example, each portion of the fluid is illuminated by at least one, preferably at least two and most preferably at least three (e.g., 3, 5, 10, 15, 20, 30, 40 or more) consecutive short duration (e.g., less than about 100 ms, preferably about 150 ps or 300 μs) pulses of high-intensity (e.g., 0.001 J/cm² to 50 J/cm², e.g., 0.01 J/cm² to 1.0 J/cm², depending on the type of fluid being treated) incoherent polychromatic light in a broad spectrum (e.g., 170 nm to 2600 nm; i.e., 1.8×10¹⁵ Hz to 1.2×10¹⁴ Hz). However, such polychromatic light may comprise wavelengths within any subset of the range of 170 nm to 2600 nm (by filtering the emitted light, for example), e.g., the energy density or fluence of the pulsed light may be concentrated within wavelengths between 170 nm and 1000 nm, between 200 nm and 500 nm, or between 200 nm and 300 nm, for example. Furthermore, it has been found that certain biological fluids are most effectively treated with many short duration pulses of polychromatic light at low fluence levels. For example, in such cases, the fluid product is illuminated with about 20, 30 or 40 or more short duration pulses at having intensities between 0.001 and 0.1 J/cm².

Broad-spectrum pulsed light (BSPL) described through this specification may also be referred to generically as “pulsed polychromatic light” or even more generically as pulsed light. Pulsed polychromatic light represents pulsed light radiation over multiple wavelengths. For example, the pulsed polychromatic light may comprise light having wavelengths between 170 nm and 2600 nm inclusive, such as between 180 nm and 1500 nm, between 180 nm and 1100 nm, between 180 nm and 300 nm, between 200 and 300 nm, between 240 and 280 nm, or between any specific wavelength range within the range of 170-2600 nm, inclusive. The choice of materials and/or spectral filters may be used produce a desired spectral range of the illumination. As is generally known, Xenon gas flashlamps produce pulsed polychromatic light having wavelengths at least from the far ultraviolet (200-300 nm), through the near ultraviolet (300-380 nm) and visible (380 nm-780 nm), to the infrared (780-1100 nm). In one example, the pulsed polychromatic light produced by these Xenon gas flashlamps is such that approximately 25% of the energy distribution is ultraviolet (UV), approximately 45% of the energy distribution is visible, and approximately 30% of the energy distribution is infrared (IR) and beyond. It is noted that the fluence or energy density at wavelengths below 200 nm is negligible, e.g., less than 1% of the total energy density. Furthermore, these percentages of energy distribution may further be adjusted. In other words, the spectral range may be shifted (e.g., by altering the voltage across the flashlamp) so that more or less energy distribution is within a certain spectral range, such as UV, visible and IR. In some embodiments it may be preferable to have a higher energy distribution in the UV range.

It is noted that although many embodiments of the invention utilize a light source 154 that provides pulsed polychromatic light (one example of which being BSPL), other embodiments of the invention use a light source 154 that provides pulses of monochromatic light, such as a pulsed laser emitting light at a specified wavelength. Thus, when referring to a fluid treatment system that uses “pulsed light”, it is meant that this pulsed light may be polychromatic or monochromatic pulsed light. It is also noted that although preferred embodiments of the invention utilize pulsed light, some embodiments utilize a light source 154 that provides continuous wave light, such as a continuous wave UV light, such as provided by Mercury gas lamps.

Thus, in general terms, the light source 154 of the fluid treatment system comprises a light source emitting light having at least one wavelength of light within a range between 170 nm and 2600 nm. For example, a pulsed polychromatic flashlamp (broad spectrum or narrow spectrum), a pulsed UV lamp, a pulsed laser, a continuous wave lamp, a continuous wave UV lamp, etc., could all serve as a light source 154 that may be used according to different embodiments of the invention.

Furthermore, in preferred embodiments, at least 0.5% (preferably at least 1% or at least 5%) of the energy density or fluence level of the pulsed polychromatic (or monochromatic) light emitted from the flashlamp 154 is concentrated at wavelengths within a range of 200 nm to 320 nm. The duration of the pulses of the pulsed light should be approximately from about 0.01 ms to about 100 ms, for example, about 10 μs to 300 μs.

In some embodiments, the fluence or intensity of the pulsed light should from 0.001 J/cm² to 50 J/cm², e.g., 1.0 J/cm² to 2.0 J/cm², depending on the fluid being treated. In embodiments where the fluid to be treated is a blood plasma derivative or other bioprocessing fluid, the fluence of the pulsed light should be carefully selected to avoid extensive protein damage while at the same time deactivate pathogens to a specified log reduction. For example, when treating biological fluids and their derivatives, such as blood, blood plasma, and blood plasma derivatives, the fluid is illuminated with pulses of light having a fluence level preferably between 0.1 and 0.6 J/cm².

As a result of such illumination, pathogens, such as microorganisms, fungus, bacteria, contained within the fluid are effectively deactivated up to a level of 6 to 7 logs reduction or more (i.e., a microbial reduction level that is commonly accepted as sterilization). Advantageously, it has been found by the inventors herein that the use of short duration, pulsed light, such as pulsed polychromatic light and broad-spectrum pulsed light (i.e., BSPL), effectively reduces the treatment time or exposure time of the treatment of fluids significantly (e.g., about 2 to 20 seconds compared to several minutes or more), increases the deactivation rate of microorganisms on objects to a level commonly accepted as sterilization (about greater than 6 logs reduction of compared to 2-4 logs reduction), in comparison to known continuous wave UV fluid treatment systems.

In many applications, biological fluids are treated primarily to deactivate pathogens without causing excessive protein damage. Thus, in these embodiments, the pulsed light treatment is configured to provide greater than 2 logs reduction, more preferably greater than 4 logs reduction and most preferably greater than 6 logs reduction is achieved with minimum protein damage. Although some of these deactivation levels fall short of what is accepted as sterilization, the pulsed light provides a significant advantage over a continuous wave UV treatment system in that pathogens and other contaminants are effectively deactivated at desired log reduction rates with minimum protein damage in a short period of time. Furthermore, the use of BSPL using Xenon flashlamps completely eliminates the problem of Mercury contamination due to broken Mercury lamps that may be encountered in such a continuous wave UV fluid treatment device, since Xenon is an inert gas which is harmless if exposed due to leakage or breaking of the Xenon flashlamp. Variants of Xenon flashlamps, such as those described in U.S. Pat. No. 6,087,783 of Eastland, et al., entitled METHOD AND APPARATUS UTLILIZING MICROWAVES TO ENHANCE ELECTRODE ARC LAMP EMISSION SPECTRA, issued Jul. 11, 2000, which is incorporated herein by reference, may also be used as an appropriate light source for the fluid treatment system 100.

Several apparatus designed to provide high-intensity, short duration pulsed incoherent polychromatic light in a broad-spectrum are described, for example, in U.S. Pat. Nos. 4,871,559 of Dunn, et al., entitled METHODS FOR PRESERVATION OF FOODSTUFFS, issued Nov. 3, 1989; 4,910,942 of Dunn, et al., entitled METHODS FOR ASEPTIC PACKAGING OF MEDICAL DEVICES, issued Mar. 27, 1990; 5,034,235 of Dunn, et al., entitled METHODS FOR PRESERVATION OF FOODSTUFFS, issued Jul. 23, 1991; 5,489,442 of Dunn, et al., entitled PROLONGATION OF SHELF LIFE IN PERISHABLE FOOD PRODUCTS, issued Feb. 6, 1996; 5,768,853 of Bushnell, et al., entitled DEACTIVATION OF MICROORGANISMS, issued Jun. 23, 1998; 5,786,598 of Clark, et al., entitled STERILIZATION OF PACKAGES AND THEIR CONTENTS USING HIGH-DENSITY, SHORT-DURATION PULSES OF INCOHERENT POLYCHROMATIC LIGHT IN A BROAD SPECTRUM, issued Jul. 28, 1998; and 5,900,211 of Dunn, et al., entitled DEACTIVATION OF ORGANISMS USING HIGH-INTENSITY PULSED POLYCHROMATIC LIGHT, issued May 4, 1999, all of which are assigned to PurePulse Technologies of San Diego, Calif. and all of which are incorporated herein by reference.

As partially shown in FIG. 3 and as more clearly illustrated in FIG. 10, the light source 154 is oriented transverse to the direction of the fluid flow. However, the light source 154 could be arranged in a different orientation, depending on the specific system configuration. Furthermore, although only one light source 154 is illustrated, more than one light source 154 could be used (e.g., one or more lamps or other light sources), depending on the length flow path, the flow rate and other requirements of the system.

In order to ensure that the light, e.g., pulsed light, emitted from the lamp assembly 150 provides the proper treatment levels, such as the proper fluence and the proper spectrum, process monitors 137 and 139 are located within the process monitor housing 138. These process monitors 137and 139 may comprise one or more of several types of optical monitoring devices, such as photodetectors, photodiodes, fiber optic probes, calorimeters, joulemeters, photomultiplier tubes (PMTs), cameras, and charged coupled device (CCD) arrays. These process monitors 137 and 139 may also be thermodetectors, such as thermocouples, thermopiles, calorimeters, and joulemeters. In one embodiment, one or more of process monitors 137 and 139 are photodetector devices that receive light emitted directly from the light source 154, as well as receive light received through the cartridge 134. Furthermore, in some embodiments, one or more of the process monitors 137 and 139 detect the ultraviolet (UV) portion of the light, while others of process monitors 137 and 139 detect full spectrum light emitted from the light source. As will be described below, the cartridge 134 includes light transmissive plates or windows (which may be generically be referred to as “light transmissive support structures”) on both sides such that the light transmits through the cartridge 134 to the treatment chamber inside. The light also transmits through the treatment chamber and the fluid through the window 135 in the cartridge 134, such that the process monitors 139 of the process monitor housing 138 can detect the light penetrating the fluid, which is also helpful to determine the absorption of light by the fluid. Additionally, process monitors 137 detect the light emitted directly from the light source 154. See FIGS. 10 and 15A-16 for further details.

The fluence level is generally adjustable by adjusting the voltage across the light source 154, e.g., flashlamp; however, it has been found that these adjustments affect the fluence or intensity profile of the emitted light over the given spectrum, i.e., a change in the voltage across the light source 154 non-uniformly changes the fluence across the given spectrum. Furthermore, the fluence received at the cartridge 134 is also adjustable by linearly adjusting the distance of the lamp assembly 150 from the cartridge 134. This provides for a uniform adjustment of the fluence without affecting its spectral intensity across the emitted spectrum. Thus, the entire lamp assembly 150 moves linearly on the lamp support plate 110 as driven by the linear slide servo drive 112. In effect, the distance from the light source 154 to the treatment chamber is adjustable. In the embodiment shown, the lamp assembly may be adjusted as much as 13 inches from the window 128 of the treatment area enclosure 104. Thus, as measured by the process monitors 137 and 139, the fluence of the emitted light is adjustable between 0.1 and 0.5 J/cm², in one embodiment, depending on the position of the lamp assembly 150 on the linear servo drive 112. Additionally, this range could be larger or smaller depending on the design and shape of the reflector 152, or modification of the size or energy of the light source 154 such as would be obvious to those skilled in the art. It is also noted that in some embodiments, the adjustment of one or more system parameters, such as fluence, fluence profile over a desired spectrum, distance of the light source 154 to the treatment chamber, voltage across the light source 154, etc., may be automatically made in response to measurements provided by the process monitors 137 and 139. In such embodiments, a controller utilizes the measurements of the process monitors 137 and 139 and automatically determines and causes the appropriate adjustments to be made in order to result in the desired system parameters as input by the user.

As will be described below, in some embodiments, the cartridge 134 contains the treatment chamber. All of the components of the fluid path, including the treatment chamber are designed to be easily removable and disposable. For example, the syringes 118 and 120, the treatment chamber, the effluent bag 140, the sample bag, and all of the tubing connecting these components are disposable. This eliminates the requirement of “cleaning” each of these components when switching between different runs of fluids. In some embodiments, the entire fluid flow path can be installed and removed as a sealed fluid flow path.

The fluid treatment system 100 is designed for adjustability of the light treatment. Such adjustability may be automatic or manual. For example, the fluence of the light treatment, the flow rate of the fluid, and the thickness of the fluid as its being treated are all adjustable. According to one example, the fluid treatment system can provide light treatment of up to 6 J/cm², and up to 10 flashes at a flow rate of 1 liter/minute. However, all of these parameters are designed to be adjustable depending on the requirements of the system and operator. Thus, in another example, with adjustments to the treatment chamber, the flow rate is scalable to 11 liters/minute or higher with similar treatment parameters. For example, the treatment can also be scaled to treatment at greater than 10 pulses (i.e., 20, 30, 40 or 50 pulses, etc.) by reflector/lamp modifications (as noted above) and/or by increasing the pulse generator power. However, it is noted that various adjustments in the pump rate, the flash rate and the relative size of various components in the fluid flow path, the flow rate is adjustable. The operator can vary the flow rate and the flash rate to any of a number of different settings. Furthermore, with minor modifications, additional, alternate pumping devices pump fluids from larger fluid sources or containers that are coupled through the cartridge 134, rather than from syringes 118 and 120, for a continuous flow and fluid treatment system.

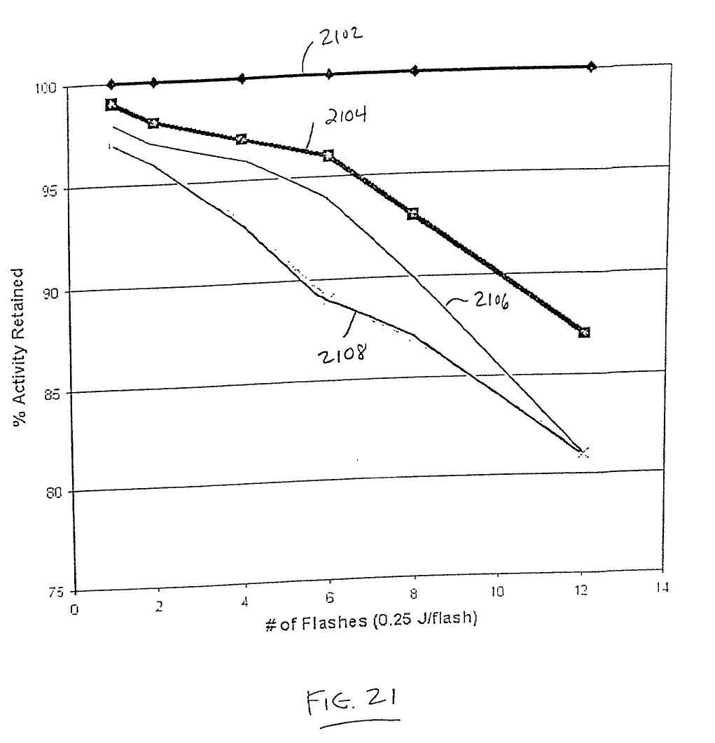

Furthermore, the fluid treatment system 100 is adapted to be coupled to a computer/controller, which provides the electronic control and processing as well as the user interface for the fluid treatment system 100. In embodiments using pulsed light, such as BSPL, an energy storage and pulse generating device is also coupled to the fluid treatment system and coupled to the flashlamp. This is more fully described with reference to FIG. 20A.

Additionally, in embodiments using pulsed light sources, such as Xenon gas flashlamps, it is known that Xenon gas flashlamps generate a significant amount of heat during extended use. However, generally, the length of time for most fluid runs using this embodiment will be very short in duration, thus, cooling means are not required. However, in a scaled up version of the fluid treatment system that is designed to run continuously and pumps fluid from a continuous source or container, cooling means are important.

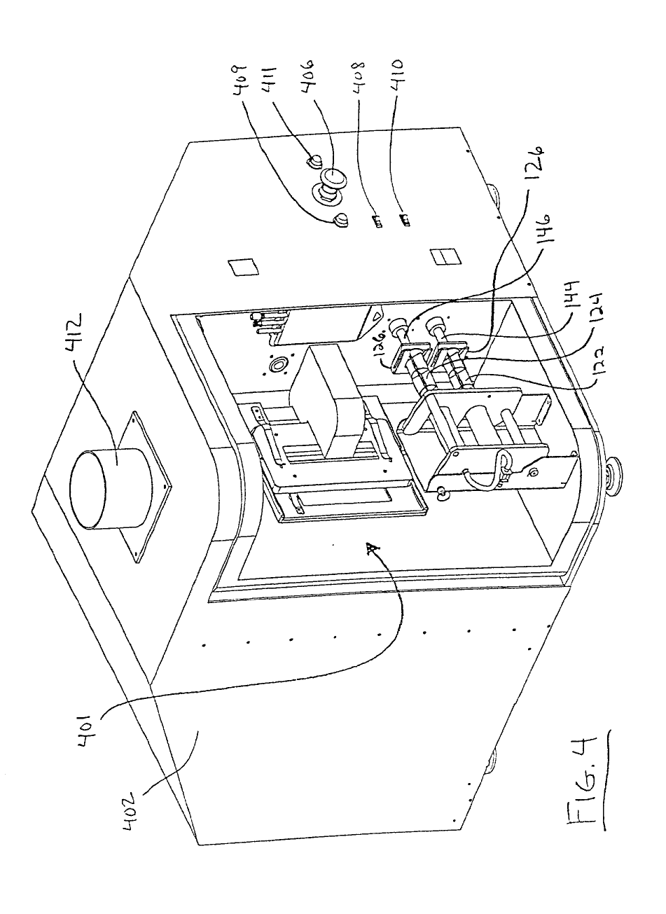

Referring next to FIG. 4, an external view is shown of the fluid treatment system of FIGS. 1-3. An enclosure 402 surrounds the fluid treatment chamber 100 such that the lamp assembly, actuator assemblies, and other electronics and controls are not accessible to the user. The enclosure 402 includes a treatment area opening 404, which allows access to the treatment area 401 including the syringes 118 and 120, the cartridge 134, the sample bag 141 and the effluent bag 140. A treatment area door (not shown) is also provided to seal off the treatment area 401 during use. Also, the treatment area 401 is sealed from the rest of the interior of the fluid treatment system 100 by the treatment area enclosure 104. Thus, any fluid spills or other accidents are confined to the treatment area 401, and will not contaminate the rest of the interior of the fluid treatment system 100. Additionally, the treatment area door is opaque to prevent the pulsed light from escaping the fluid treatment system during use. The enclosure 402 also includes user controls, such as an emergency power off switch 406 and indicator lights 409 and 411. Additionally, also provided are toggle buttons 408 and 410, which are used to adjust the linear position of the linear actuator 144 and 146 either left or right in order that they can properly retain the plunger heads of the syringe plungers 122 and 124. This is because, the heads of the syringe plungers 122 and 124 extend a variable distance from the body of syringes 118 and 120. Since the plunger heads are to be held by the brackets 126 at the end of the linear actuators 144 and 146, the toggle buttons 408 and 410 move the bracket to the left or right. Thus, the plunger heads will align within the brackets 126. Furthermore, a fan cover 412 is also shown. The fan cover 412 heat and/or ozone to be pulled from the interior of the fluid treatment system to the exterior by a fan underneath the fan cover 412.

Referring next to FIG. 5, a perspective view is shown of the syringe mount assembly 116 of FIGS. 1-3 according to one embodiment of the invention. In order to load syringes, e.g., syringes 118 and 120 of FIG. 1, a syringe pump mount plate 502 (also referred to generically as a fluid container holder) rotates outward relative to a syringe pump mount bracket 504 about bar 506. The syringe pump mount plate 502 includes slots 508 and 510 for receiving syringes 118 and 120, respectively. Once positioned in the slots 508 and 510, the syringe pump mount plate 502 is rotated back flush with the syringe pump mount bracket 504. Pushpin 512 is inserted through hole 514 of the syringe pump mount bracket 504 and hole 516 of the syringe pump mount plate 502 to lock the syringe mount assembly 116 in position.

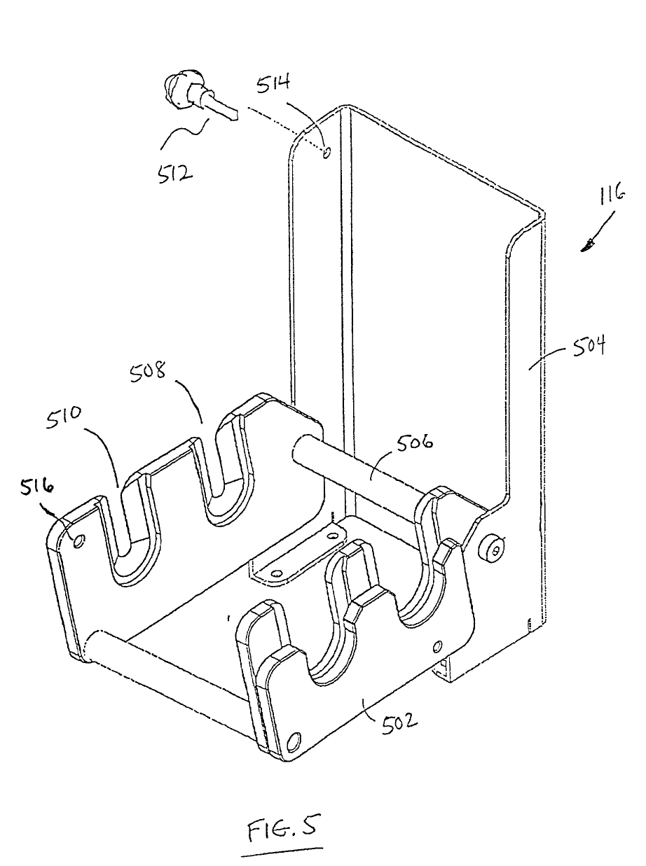

Referring next to FIG. 6, a schematic view is shown of one emboidment of the fluid flow path components of the fluid treatment system of FIGS. 1-3. Shown are the syringes 118 and 120 (each of which may be generically referred to as “fluid container portions” of a fluid flow path for use in a generic fluid treatment system) including tubes 602 and 604, respectively. Tubes 602 and 604 are connected at Y-fitting 606. Alternatively, Y-fitting 606 is a T-fitting, as is illustrated in FIGS. 1 and 3. A T-fitting is preferable since the T-fitting can be directly coupled to one of the syringes (e.g., syringe 118 of FIG. 1) such that tube 602 can be eliminated or its length shortened. Tube 608 (also referred to as the supply conduit or input conduit) couples the Y-fitting 606 (or alternatively, T-fitting or other fitting) to an input of a treatment chamber 610. The treatment chamber 610 may also be referred to generically as a “treatment chamber portion” of a fluid flow path. An output of the treatment chamber 610 is coupled to tube 612 (also referred to as the output conduit), which splits at Y-fitting 614 into tubes 616 and 618, which are connected to a sample bag 141 and the effluent bag 140, respectively. The sample bag 141 and the effluent bag 140 can be generically referred to as fluid container portions or fluid collector portions of the fluid flow path. In order to easily connect the treatment chamber 610 in-line, quick disconnect 622 is optionally provided in tube 608 and quick disconnect 624 is provided in tube 612. These quick disconnects 622 and 624 may be any quick disconnects as known in the art, such as CDC quick disconnects produced by Colder Products Company of St. Paul, Minn., USA or other luer quick disconnects available from Value Plastics, Inc. of Fort Collins, Colo., USA, as known in the art. Furthermore, solenoid valves 626 and 628 (e.g., pinch valves) control the flow of fluids into the sample bag 141 and the effluent bag 140, respectively.

Additionally, in order to monitor the pressure and temperature of the fluid flow, pressure transducer 632 and thermocouple 630 are coupled the input of the treatment chamber 610, e.g., coupled to tube 608.

Additionally, pressure transducer 636 and thermocouple 634 are coupled at the output of the treatment chamber 610, e.g., coupled to tube 612. These pressure transducers and thermocouple provide an electrical signal to be transmitted to a process controller of the system. Thus, the system is able to measure the pressure of the fluid flow at the input and the output of the treatment chamber, as well as monitor any changes in the temperature of the fluid flow due to the light treatment. It is noted that Xenon gas flashlamps and other pulsed light sources may generate significant heat, which may increase the temperature of the fluid. Thus, depending on the sensitivity to heat of the fluid being tested, the fluence of the light source 154 may be adjusted (e.g., by adjusting the distance between the light source 154 and the treatment chamber 610) in response to the measurements taken by the pressure transducers and thermocouples. It is noted that the pressure transducers 632 and 636 and thermocouples 630 and 634 may also be referred to generically as process monitors, since they are used to monitor the fluid flow.

In operation, syringe 118 contains the fluid to be delivered, i.e., contains the inoculated or contaminated fluid, while syringe 120 contains either uninoculated fluid or WFI (water for injection), or other solutions as described above. Actuator devices or pumps (e.g., actuator assembly 106 including linear actuator 144, or other pumping devices) operate independently or at the same time to apply forces, e.g., F1 and F2, to the plungers 122 and 124 of the syringes 118 and 120. This causes the fluids within one or more of the syringes 118 and 120 to be forced into the tubes. For example, the fluid in syringe 118 is forced into tube 602, through Y-fitting 606, through tube 608 and through the treatment chamber 610 or treatment chamber at a desired flow rate. The flow rate is dictated by the syringe barrel diameter and the linear actuator velocity, which is set by the operator and coordinated with the flash rate of the flashlamp 154. These actuator assemblies are under the control of electronics within the fluid treatment system.

As the fluid passes through the treatment chamber 610, the fluid is exposed to the light treatment, e.g., the fluid is exposed to one or more flashes of pulsed light emitted from light source 154. Also included is the reflector 152 positioned behind the light source 154 and is shaped to project a fluence pattern toward the treatment chamber 610. In one embodiment, the light source 154 is a Xenon gas flashlamp which emits BSPL, as described above. The fluence of the light received at the treatment chamber 610 is adjustable by adjusting the power to the light source 154 and/or by adjusting the linear distance between the light source 154 and the treatment chamber 610. It is noted that a linear distance adjustment is preferred since it provides for a uniform adjustment of the fluence across the full spectrum of the emitted light. It is noted that although only one light source is shown, the system may include more than one light source or lamp.

The fluid continues to flow out of the treatment chamber 610, through tube 612, Y-fitting 614, and into one or both of the sample bag 141 and the effluent bag 140, via tubes 616 and 618, respectively. The fluid flow into the sample bag 141 and effluent bag 140 is controlled by the solenoid valves 626 and 628. During most of the fluid run, solenoid valve 628 is open and solenoid valve 626 is closed such that the fluid is directed to the effluent bag 140. Thus, the effluent bag 140 contains a mixture of the decontaminated fluid product and fluids from syringe 120, e.g., water for injection or other solutions. Alternatively, the effluent bag 140 may contain only the fluid to be treated in the event both syringes 118 and 120 contain the same fluid. In order to collect a clean, usable sample, solenoid valve 626 is opened while solenoid 628 is closed to collect a predetermined amount (set by the operator) of fluid within the sample bag 141 for testing and evaluation or use.

Generally, the treatment chamber 610 may be a flexible or rigid structure having a given geometry. According to several embodiments, the treatment chamber 610 is generally a substantially flat sheet-like treatment chamber. The treatment chamber may be disposable or reusable. The treatment chamber 610 may also be a flexible bag-like material or a rigidly shaped material. In some embodiments, the treatment chamber is a substantially tubular structure that may be flexible or rigid. In some embodiments, the treatment chamber 610 is generally held within a cartridge, such as shown in FIGS. 1-3; however, in alternate embodiments, the cartridge is not required, such as shown in FIG. 11. Thus, in the alternate embodiments, the treatment chamber is simply positioned in front of the lamp assembly 150 for treatment. In embodiments using a cartridge, the cartridge restrains the treatment chamber 610 between two light transmissive support structures or plates separated by a specified distance. Thus, in some embodiments, the flow of fluid within the treatment chamber 610 is a substantially flat laminar flow having an adjustable thickness and an adjustable width. However, it is noted that the flow may be characterized as flat, laminar, uniform, tubular, turbulent or any other flow as understood in the art. The thickness is adjustable by using an adjustment mechanism that varies the specified thickness. The width is adjustable in the selection of the appropriate treatment chamber. For example, the operator may have a choice between many differently sized treatment chambers having different widths depending on the manufacturing specifications.

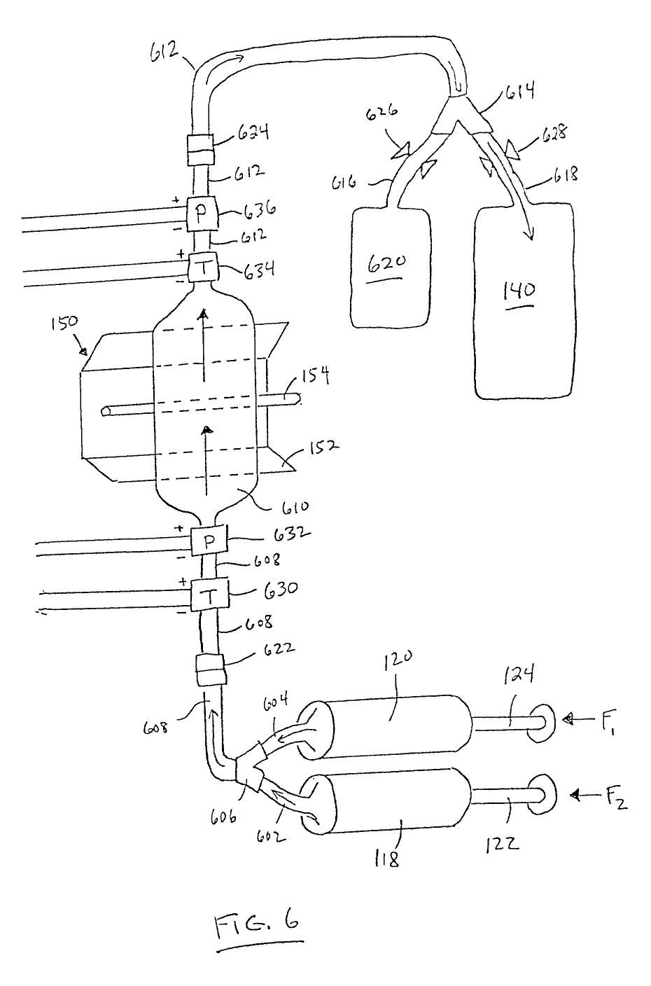

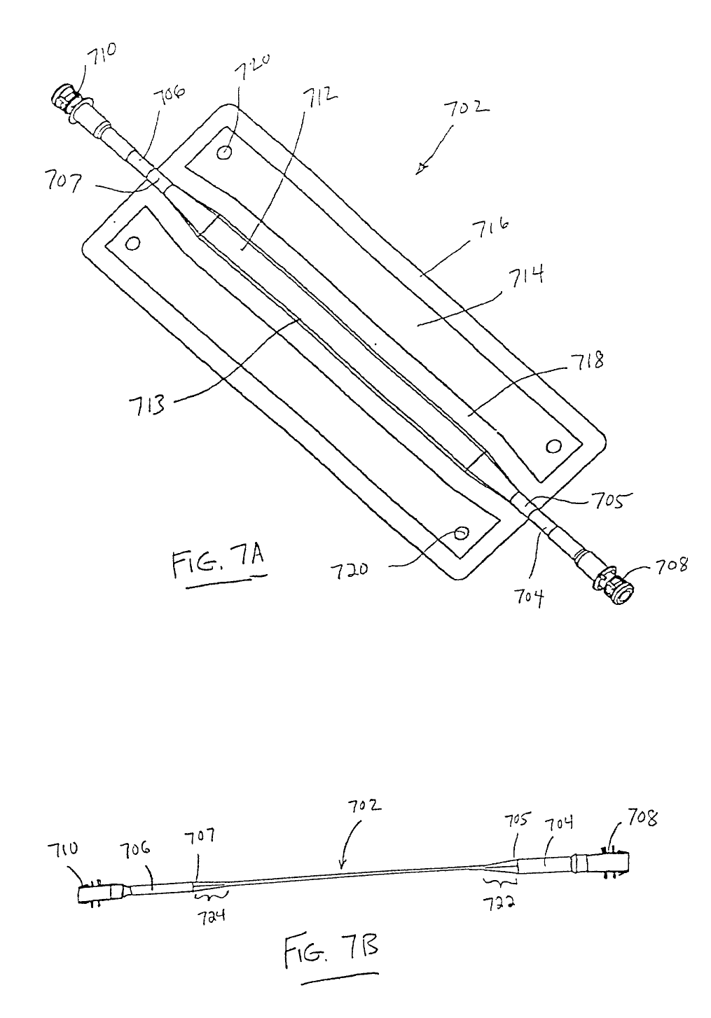

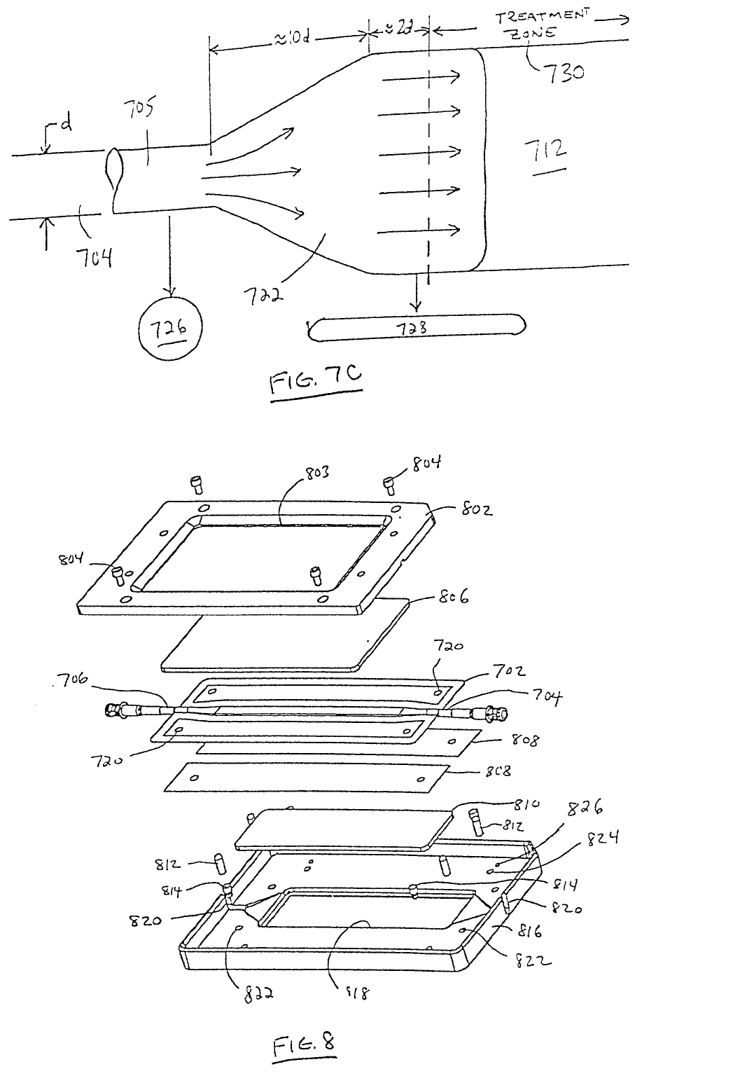

Generally, the treatment chamber 610 is light transmissive. In some embodiments, at least a portion of the treatment chamber is transmissive to at least 1% of light having at least one wavelength between 170 and 2600 nm. For example, the treatment chamber 610 is made of materials transmissive at least portions of the light emitted by the light source 154, e.g., FEP (flourinated ethylene-propylene perfluoro (ethylene-propylene)), EVA (ethylene vinyl acetate), PTFE (polytetrafluoroethylene), PFA (perfluoro (alkoxy alkane)), ethyl vinyl alcohol, polyvinylidene fluoride (PVDF), polyvinyllidine chloride (PVDC): Saran, and polyamides, such as nylon and polychlorotrifluoroethylene (PCTFE): Aclar. Thus, in some embodiments, the treatment chamber 610 is made of materials such as polymers, polyolefins, fluorinated polymers, halogenated polymers, polyamides, nylons, plastics, or combinations thereof. Various embodiments of the treatment chamber 610 and the cartridge are described further below, for example, with reference to FIGS. 7A, 7B, 12, 13, and 14, although it is appreciated that the treatment chamber may take many forms other than those specifically described in FIGS. 7A, 7B, 12, 13 and 14.

In one embodiment, the entire fluid flow path is sealed and removable from the fluid treatment system. In this embodiment, the fluid flow path may be defined as having a first fluid container portion, e.g., one or both of the syringes 118 and 120, a treatment chamber portion, e.g., the treatment chamber 610, and a second fluid container portion, e.g., one or both of the sample bag 141 and the effluent bag 140. The first fluid container portion contains the fluid to be treated with the light treatment. The fluid in the first fluid container portion is flowed through the treatment chamber portion and illuminating with light. The treated fluid is collected in the second fluid container portion. Advantageously in this embodiment, the first fluid container portion is sealingly coupled to an input of the treatment chamber portion (e.g., using flexible tubing and connectors) and the second fluid container portion is sealingly coupled to an output of the treatment chamber portion (e.g., using flexible tubing and connectors). In this embodiment, the entire fluid flow path may be pre-sterilized and contain the fluid to be treated. The entire fluid flow path may be inserted into the fluid treatment system (e.g., the fluid treatment system 100) and removed once the light treatment is completed. Once the treated fluid or treated sample is removed, the entire fluid flow path may then be discarded and replaced with another fluid flow path; thus, eliminating the need to sterilize the fluid flow path after each use.

Furthermore, in some embodiments, many components of the fluid flow path are designed of inexpensive materials, such as plastics, nylons, polymers, or combinations thereof. Many of these components may also be made of generally flexible materials. It is noted that although the entire fluid flow path may be made sealed and removable from the fluid treatment system in some embodiments, the fluid flow path is not required to be installed as a sealed fluid flow path. For example, one or more components may be inserted separately into the fluid treatment system and then coupled and sealed together. In another example, the entire fluid flow path may be coupled and sealed together and then inserted into the fluid treatment system.

Furthermore, sealed fluid flow path may be embodied in any number of geometries and includes for example, a first container portion that contains a fluid to be treated, a treatment chamber portion coupled to the first container portion that is adapted to have the fluid flowed therethrough and a second container portion coupled to the treatment chamber portion that is adapted to receive the fluid that is flowed through the treatment chamber portion. The fluid may be flowed through the treatment chamber portion using a pump or other device or by any means to cause the fluid to flow from one portion to another, for example, even through the use of gravity. While the fluid is being flowed through the treatment chamber portion, the fluid is treated with light from the light source. The different portions may be coupled to each other via tubing or connectors as illustrated, or in other embodiments, the first container portion, the second container portion and the treatment chamber portion are one integral structure. Furthermore, in some embodiments, the sealed fluid flow path may be made of any of the materials listed above and may be flexible or rigid. It is also noted that in some embodiments, the fluid to be treated may initially not be present in the first container portion, but is injected or inserted into the first container portion prior to being flowed through the treatment chamber portion. It is also noted that the flow of the fluid through the treatment chamber 610 may take a variety of forms. For example, depending on the geometry of the treatment chamber, the fluid may flow therethrough in a laminar flow, a flat flow, a tubular flow, a uniform flow, a non-uniform flow, and a turbulent flow to allow mixing, etc.

Referring next to FIG. 7A, a perspective view is shown of one embodiment of the treatment chamber of FIG. 6. Illustrated is the treatment chamber 702 including an input tube 704 (or supply conduit) coupled to an input port 705, an output tube 706 (or output conduit) coupled to an output port 707, each having a respective quick disconnect 708 and 710. The input tube 704 and the output tube 706 are round tubes coupled to the input and output ports 705 and 707. The input and output ports 705 and 707 taper into a flow chamber 712 of the treatment chamber 702. It is noted that in preferred embodiments, the taper from the input and output ports 705 and 707 to the flow chamber 712 should be designed to uniformly translate the generally circular cross sectional flow of the fluid through the tubes to the substantially laminar flow profile through the flow chamber 712. This is further illustrated with reference to FIG. 7C. However, it is noted that the taper from the input and output ports 705 and 707 may be made to designed to minimize dead spots or stagnation and to generally maintain a substantially uniform flow. In other embodiments, the flow through the flow chamber 712 may be designed to be a turbulent flow such that the fluid is mixed as it is flowed through the flow chamber.

The flow chamber 712 extends from the input port 705 to the output port 707. The body portion 714 of the treatment chamber 702 is generally formed using multiple sheets of a light transmissive material, such as a polymer, polyolefin, fluorinated polymer, halogenated polymer, polyamide, nylon, plastic, or combinations thereof. Thus, by way of example, FEP, EVA, PTFE, PFA, PVDF and PCTFE may be used for the body portion 714. These two sheets are placed on top of each other and sealed together at the exterior edges 716 and at the boundary 718 to the flow chamber 712. For example, the sheets of material are welded (e.g., radio frequency (RF) welded), or other wise bonded to each other to form the treatment chamber 702. Thus, the treatment chamber 702 is generally flat and flexible, having a flow chamber 712 formed therethrough.

In some embodiments, prior to bonding or attaching the sheets together, a slight preform 713 is formed in each sheet of material proximate to the boundary of the flow chamber 712. The preform 713 may be a slight bend or other deforming feature. This preform allows the flexible sheets to form the flow chamber more naturally without causing creasing along the edge of the flow chamber as the fluid fills up and passes through the flow chamber 712. However, even with the preform, the flow chamber is substantially flat without the presence of a fluid flowing therethrough.

In operation, the fluid is forced through the input port 705 into the flow chamber 712 and out through the output port 707 at a controlled rate. As the fluid product flows through the flow chamber 712, the volume of the flow chamber expands, i.e., the flow chamber fills up to form a generally flattened elliptical tubular structure. However, the thickness of the flow chamber 712 is generally not uniform across the width of the flow chamber 712. For example, the flow chamber 712 is slightly wider at the center in comparison to the edges across the width of the flow chamber 712. Additionally, the thickness of the material of the body portion 714 that forms the flow chamber 712 is designed to be able to withstand the pressure of the fluid as it is pumped or other wise forced through the flow chamber 712.

In preferred embodiments, the treatment chamber 702 is positioned against a structure that is at least partially light transmissive, e.g., positioned within the cartridge as described above. In order to align the treatment chamber 702 within the cartridge, holes 720 are punched in the body portion 714 through which alignment pins of the cartridge or other retaining assembly pass. It is noted that these holes 720 may be referred to generically as “alignment features” and the alignment pins may be referred to generically as “corresponding alignment features”. Other types of alignment features and corresponding alignment features may include tapers, wedges, ridges, key in slots, etc.

As described further below, several embodiments include one or more light transmissive support structures, e.g., plates or windows, positioned against the treatment chamber 702. The one or more support structures effectively define one or more dimensional boundaries of the flow chamber 712; thus, the one or more light transmissive support structures define one or more dimensional boundaries of the treatment zone or treatment volume. For example, if the treatment chamber 702 is held against a single plate or window, the single plate or window defines one dimensional boundary of the flow chamber 712. In the case of two plates or windows, the treatment chamber 702 is sandwiched between the two plates, i.e., the two plates define two dimensional boundaries of the flow chamber 712. These plates or windows effectively flatten out the flow chamber 712 once the flow chamber is filled with fluid to provide a laminar fluid flow through the flow chamber 712 for substantially uniform light treatment. Depending on the shape of the one or more plates or windows, the thickness therebetween may or may not be uniform; thus, the fluid flow may or may not have a uniform thickness throughout the length of the flow chamber 712. The distance between the two plates or windows can be controlled, such that the flow chamber 712 has an adjustable fluid thickness. In some embodiments, the fluid flow is substantially uniform across its width and along the length of the flow chamber 712. It is noted that the one or more support structures may comprise flat or curved plates, and at least portions of which may be transmissive to at least a portion of the light treatment. In embodiments where the plates are curved, the curvature of the two plates may be the same or different depending upon the embodiment. It is noted that the one or more plates may be referred to generically as a “treatment chamber support structure” or “treatment zone support structure” that defines one or more dimensional boundaries of the flow chamber 712 or treatment zone or treatment volume. It is also noted that in alternate embodiments, the treatment chamber 702 itself may be positioned in front of one or more light sources without necessarily being positioned within or against one or more light transmissive support structures, e.g., plates or windows. In some embodiments described below, the treatment chamber is held within a specially designed cartridge. In some embodiments, the treatment chamber 702 resembles a liner-like structure to the support structure (e.g., the one or more plates or windows or the cartridge).

Advantageously, the treatment chamber 702 is designed to be light transmissive to at least a portion of the light emitted from the light source 154. Furthermore, the treatment chamber 702 is easily manufactured such that it is disposable after use. The treatment chamber 702 is simply removed at the quick disconnects 708 and 710 and replaced for the next fluid treatment. This eliminates the requirement of having to clean out or flush the treatment chamber 610 when switching between different types or runs of fluids. In some embodiments, the entire fluid flow path is disposable. For example, the treatment chamber 702 along with the syringes, the tubing, and the sample bag and the effluent bag are all removed and replaced after each use. Advantageously, there is not need to clean out these components since they are replaced by pre-sterilized components for the next run.

This treatment chamber is a departure from known light treatment devices. In known light treatment fluid devices, a volume is defined within the device that is a treatment volume. The fluids, typically water, are passed through the treatment volume at a low flow rate and treated with light, such as continuous wave ultraviolet light. The treatment volume is defined by a container that allows the fluid to flow therethrough. This treatment chamber is a rigid structure that is designed for multiple uses and must be cleaned out prior to treating different fluids. Such treatment chambers are commonly made of a rigid quartz, or similar light transmissive, material. Manufacturing a quartz container can be expensive and time consuming. Thus, replacing such a quartz material treatment chamber after each use would be prohibitively expensive. Furthermore, such treatment chambers are rigid in order to adequately contain the fluid product.

In contrast, the treatment chamber of this and other embodiments of the invention is disposable and flexible. The dimensional boundaries are not rigidly set and may be affected by positioning the treatment chamber against the appropriate support structure. Applicants are not aware of other flexible treatment chambers. A sealed flexible bag containing a fluid may be treated within a treatment device; however, the fluid is static within such as bag and is not flowed from one portion to another portion. The flexible treatment chamber of several embodiments of the invention does not initially contain the fluid. The fluid is pumped through the treatment chamber 702 from the input tube 704 (supply conduit) to the output tube 706 (output conduit). As the fluid is flowed through the treatment chamber, the fluid is treated with light. Using the proper flexible and light transmissive materials, the treatment chamber 702 is inexpensive to manufacture and is easily replaceable. For example, if such a treatment chamber were made of a rigid quartz material, such a treatment chamber would be more expensive to manufacture and would have to be cleaned after each use. Furthermore, it has been found that adhesives used to manufacture such a quartz treatment chamber react negatively with certain types of biological fluids and blood plasma derivatives. Advantageously, because the treatment chamber 702 is disposable, the treatment chamber 702 does not have to be cleaned, it is simply replaced after usage.

It is noted that depending on the desired flow rate and the type of fluid product to be pumped through the treatment chamber 702, the dimensions of the treatment chamber 702 may be altered. For example, the treatment chamber could be made longer or wider. The flow chamber 712 could be made wider or narrow, as well.

Referring next to FIG. 7B, a side view is shown of the treatment chamber 702 of FIG. 7A. As can be seen, the treatment chamber 702, the body portion 714, including the exterior edges 716, the flow chamber 712 and the boundary 718 are substantially flat, even with the presence of the preforms (see FIG. 7A) formed in the flow chamber 712. As shown at taper sections 722 and 724, the flow chamber 712 tapers outward to form the input port 705 and the output port 707, respectively. Also illustrated are input and output tubes 704 and 706 which couple to quick disconnects 708 and 710. As shown, fluid is not flowing through the flow chamber 712. Advantageously, the treatment chamber 712 provides a thin fluid flow path the width of the flow chamber 712. Furthermore, in this embodiment, the treatment chamber is designed to be a flexible flat treatment chamber.

Referring next to FIG. 7C, a schematic view of a transition from a circular flow profile to a substantially flat profile at the input and output of the treatment chamber of FIGS. 7A and 7B according to another embodiment of the invention. At the input port and the output port 705 and 707 of the treatment chamber of FIGS. 7A and 7B, the fluid flow has a generally circular cross sectional profile 726 (defined by the diameter d of the input and output tubes). However, when the treatment chamber is positioned between two plates, for example, light transmissive plates, the flow chamber 712 has a relatively flat cross sectional profile 728 with an adjustable thickness (depending upon the spacing of the two plates). Thus, according to this embodiment, the circular flow profile is to be transitioned or redistributed to a substantially flat flow profile. This is accomplished in the taper at taper section 722 (and 724). In preferred embodiments, it is desired that the transition take place such that the laminar fluid flow through the flow chamber 712 has substantially the same velocity across the width of the fluid flow. Thus, by carefully designing the taper section 722, the fluid flow being illuminated (e.g., within the treatment zone 730 portion of the flow chamber 712) has a substantially uniform, stream-lined velocity across its width.

Thus, the taper section 722 (and 724) is carefully configured to provide a smooth transition from the circular to the substantially flat profile. According to one embodiment, the length of the taper section 722 is approximately equal to 10 times the diameter of the circular fluid profile entering the taper section 722. Once the fluid flow exits the taper section 722, according to one embodiment, a distance of approximately 2 times the diameter of the circular fluid profile, is required to streamline the relative velocities of portions of the fluid flow in-line, such that when the fluid flow enters the treatment zone 730, the fluid flow will effectively be translated to a substantially laminar flow having substantially the same velocity across the width of the flow chamber 712, i.e., the fluid flow is a substantially uniform, streamlined velocity. A similar taper is formed at the taper section 724 at the output port of the treatment chamber to redistribute the laminar flow back to a circular flow, preferably having the same distance from the treatment zone 730 to the beginning of the taper section 724 and from the beginning of the taper section 724 to the output port 707.

Advantageously, by appropriately sizing the taper sections 722 and 724, dead spaces, stagnation and eddies are prevented from forming in the treatment zone 730 of the flow chamber 712, i.e., a substantially uniform fluid flow results. Thus, a smooth transition from the tube to the flow chamber 712 occurs at the input port 705. Also, the transition back to the substantially circular flow at the output port 707 is smooth in order to not disrupt the flow within the treatment zone 730. It is also noted that in some embodiments, the flow through the treatment chamber or treatment zone may be designed so as to not be uniform and even turbulent.

Referring next to FIG. 8, an exploded view is shown of one embodiment of the cartridge as shown in FIGS. 1-3 illustrating the treatment chamber of FIG. 7 positioned therein. Illustrated is a cartridge 800 including a cartridge top 802, cartridge top opening 803, screws 804, a first window 806 (also referred to as a first light transmissive window or plate or generically, a light transmissive support structure), the treatment chamber 702, opaque pieces 808, a second window 810 (also referred to as a second light transmissive window or generically as a plate portion or support structure portion), alignment pins 812 (referred to generically as alignment features), spacers 814, a cartridge bottom 816, alignment pin holes 822 (referred to generically as corresponding alignment features), spacer holes 824, threaded holes 826, a cartridge bottom opening 818 and slots 820. It is noted that the cartridge top 802 and the cartridge bottom 816 may be referred to generically as “parts” of a cartridge body.

The first window 806 is attached or adhered within the opening 803 of the cartridge top 802. The first window 806 is designed to be transmissive to at least a portion of the light treatment. The second window 810 is attached or adhered in position within the cartridge bottom opening 818 and is also transmissive to at least a portion of the light treatment. For example, the first window 806 and the second window 810 are transmissive to at least 1% of light having at least one wavelength within the range of 170 to 2600 nm. The first window 806 and the second window 810 are preferably made of quartz or similar material. The spacers 814 and the alignment pins 812 are attached to the cartridge bottom 816 within spacer holes 824 and alignment pin holes 822, respectively. Optionally, opaque pieces 808 are positioned on top of the cartridge bottom 816 such that they fit over the alignment pins 812 and block light from the sides so that the light entering through the second window 810 (to process monitors, such as a fiber probe or photodetector) is the light transmitted through the flow chamber. Next, the treatment chamber 702 is positioned over the opaque pieces 808 within the cartridge bottom 816. The alignment pins 812 extend through the holes 720 of the treatment chamber 702 to ensure alignment. Next, the cartridge top is positioned over the treatment chamber 702 and the screws 804 are threaded into the threaded holes 8826 of the cartridge bottom 816 to the desired tightness. Using the spacers 814 (e.g., 1-5 mm thick), a variable thickness between the first window 806 and the second window 810 can be achieved. Note that the input tube 704 and the output tube 706 fit within the respective slots 820 of the cartridge 800. It is noted that the second window 810 is not required to light transmissive. In embodiments where the second window 810 or plate portion is not light transmissive, the second window could be integrated into the cartridge bottom 816. It is preferably light transmissive to enable measurement of the light treatment that transmits through the fluid and to avoid reflections back into the treatment chamber.

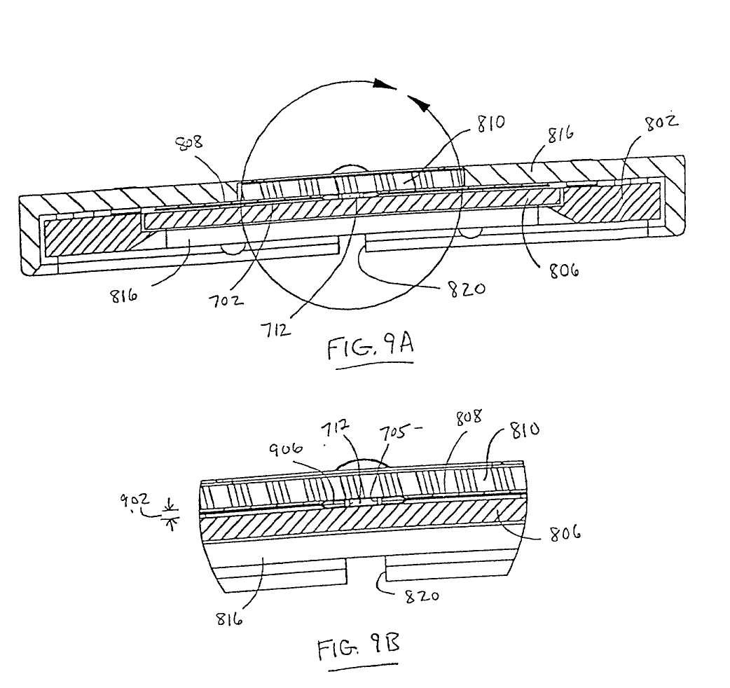

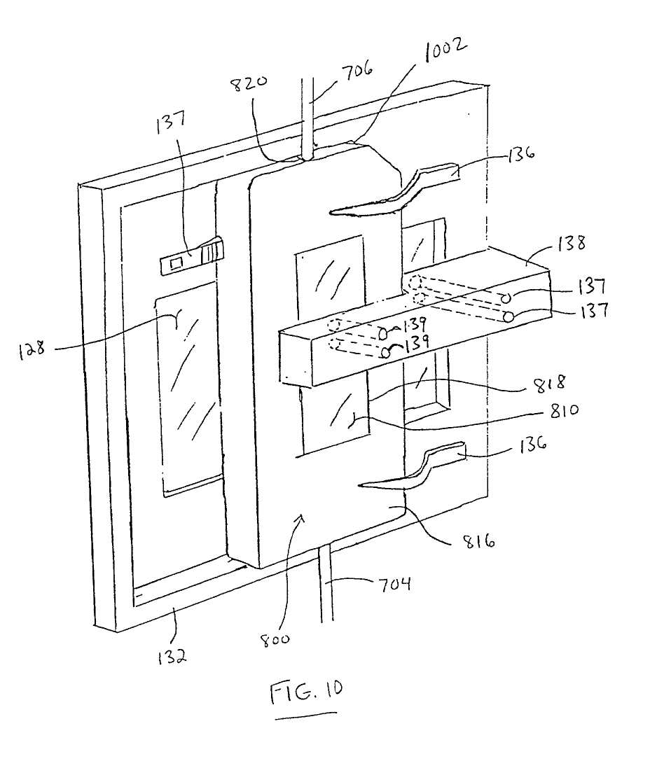

Referring next to FIGS. 9A and 9B, cross sectional views are shown of the cartridge of FIG. 8 containing the treatment chamber of FIGS. 7A-7B according to one embodiment of the invention. The view of FIG. 9A is a full cross sectional view across the width of the cartridge 800, while the view of FIG. 9B is an enlarged view of the portion of the view of FIG. 9A illustrating the flow chamber. As illustrated, the treatment chamber 702 is held between the first window 806 (or plate) and the second window 810 (or plate). As fluid flows through the flow chamber 712, the flow chamber expands or fills up. However, in this embodiment, since the flow chamber 712 is positioned between rigid plates, i.e., the first and second windows 806 and 808, the flow chamber 712 is forced to have a substantially uniform thickness 902 across the width of the flow chamber 712 and through the length of the flow chamber 712. As such, advantageously, the fluid flows through the flow chamber 712 substantially uniformly such that the light treatment penetrates all portions of the fluid to the same extent. In some embodiments, it is important to ensure that all portions of the fluid are treated equally, rather than some portions of the flow chamber being thicker than other portions, in the event such a flow chamber 712 were tubular. Also illustrated in the cross sectional view of FIG. 9B is the input port 705 (or alternatively, the output port 707). Line 906 represents the tapering from the input port 705 to the full width of the flow chamber 712.

It is noted that in alternate embodiments, the two support structures or plates, e.g., the first window 806 and the second window 810 may be curved or flat (as illustrated) and each may have a separate physical shape.

In some embodiments, the cartridge is not used, instead the treatment chamber 702 is mounted or positioned in front of a lamp assembly. In such alternative embodiments, the thickness of the flow chamber 712 may vary across the width of the flow chamber 712. Advantageously, by using the cartridge, the flow chamber 712 is sandwiched between two plates. Thus, this embodiment of a treatment chamber support structure restrains the flow chamber 712 such that it defines at least one dimensional boundary of the flow chamber 712, i.e., the top and bottom surfaces. At least one of these structures must be light transmissive, while the second plate may or may not be light transmissive. Thus, the first window 806 is light transmissive while the second window 810 is not required to be light transmissive. However, in preferred embodiments, the second window 810 is light transmissive to allow for photodetectors to view and measure the light penetrating through the treatment chamber and the fluid product and also to prevent reflected light from entering back into the treatment chamber.

It is noted that in some embodiments, the treatment chamber 702 does not have to be positioned within a cartridge for the flow chamber 712 to be substantially flattened. For example, the treatment chamber 702 (including the flow chamber 712) may be held or positioned against one or more support structures, e.g., positioned against one plate or sandwiched between two plates in order to sandwich the flow chamber 712 therebetween such that the flow chamber (or generically, the treatment zone) is restrained by the support structures (in this case, plates or windows). Thus, in these embodiments, the treatment chamber support structure defines one or more dimensional boundaries of the flow chamber 712. At least one of these plates is light transmissive, preferably both plates. For example, one of the plates may be window 128. It is also noted that in alternate embodiments the treatment chamber support structure may be such that the thickness of the flow chamber is variable along its length, i.e., not necessarily a flat or plate-like structure.

Referring next to FIG. 10, a perspective view is shown of the cartridge of FIG. 8 as positioned within the cartridge registration plate of the fluid treatment system of FIGS. 1-3 according to one embodiment of the invention. As seen, the cartridge 800 containing the treatment chamber, is positioned within the cartridge registration plate 132 of the treatment area enclosure 104. As such, the cartridge 800 is registered within the cartridge registration plate 132. The cartridge 800 slides underneath the process monitor housing 138 until it is flush with edge 1002 of the cartridge registration plate. The cartridge lock clips 136 and the cartridge retaining clip 137 hold the cartridge 800 in place. Thus, the cartridge inserts into the cartridge registration plate 132. Furthermore, the cartridge is thick enough such that the input tube 704 and the output tube 706 extend from the slots 820 without bending.

Also illustrated are the process monitors 137 and 139 that measure the light, e.g., pulsed light. As can be seen process monitors 139 view light from the light source that passes through the cartridge 800 and the treatment chamber, while process monitors 137 view the light emitted directly from the light source having passed through the window 128. It is noted that these process monitors 137 and 139 are shown from the back. The process monitors 137 and 139 face toward the light source located on the opposite side of the window 128. In some embodiments, one or more of the process monitors 137 and 139 may be optical detectors, such as photodiodes or other photodetectors as known in the art, while in other embodiments, the one or more of the process monitors 139 and 139 may be fiber probes coupled to fiber optic cabling that extends from the process monitor housing to the electronics and control portion of an optical monitoring system. In other embodiments, one or more of the process monitors 137 and 139 may be pressure transducers or thermopiles, as are known in the art.

Referring next to FIG. 11, another embodiment of the fluid treatment system of FIGS. 1-3 is shown. Several of the components of the fluid treatment system of FIGS. 1-3 are the same as previously described. In this embodiment, the cartridge 134 is not used to contain the treatment chamber 702. The treatment chamber 702 (i.e., one embodiment of the treatment chamber 610 of FIG. 6) is simply positioned within the cartridge registration plate 132 (which may be generically referred to as a treatment chamber mounting device or treatment chamber support structure) and held in place with clips. Thus, as described above, the cartridge is not used in all embodiments; however, the cartridge is preferred since it restrains the flow chamber of the flexible, light transmissive treatment chamber 702 in order to define at least one dimensional boundary of the treatment chamber. In preferred embodiments, the cartridge provides for a substantially uniform thickness of the fluid flow along the length of the treatment chamber. Furthermore, it is noted that a support structure or plate (preferably light transmissive) may be positioned to restrain or sandwich the flow chamber of the treatment chamber 702 against the window 128 (e.g., using clips or adjustable screws with spacers) to provide a substantially flat laminar flow (or curved or turbulent flow, as desired) through the treatment chamber 702 without requiring that the treatment chamber be within a cartridge. In some embodiments, the clips press the flow chamber against the window 128; thus, the window 128 becomes the support structure that defines one dimensional boundary of the flow chamber of the treatment chamber 702. In embodiments where the treatment chamber is held between two plates or windows, the two plates or windows become a support structure that defines two dimensional boundaries of the flow chamber. Again, advantageously, the entire treatment chamber, as well as all of the components in the fluid flow path, are disposable upon completion of the fluid run.

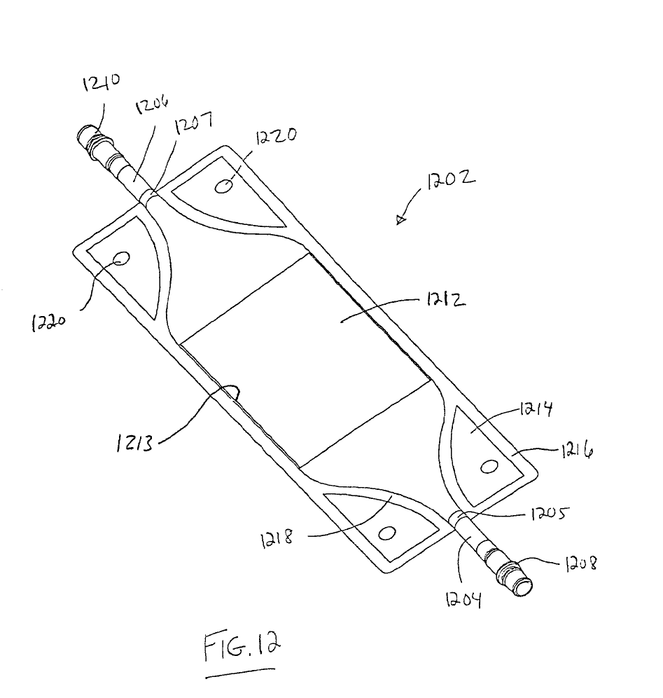

Referring next to FIG. 12, a perspective view is shown of a flat, disposable treatment chamber that may be used in the fluid treatment system of FIGS. 1-3 in accordance with another embodiment of the invention. Shown is the treatment chamber 1202 including an input tube 1204 coupled to an input port 1205, an output tube 1206 coupled to an output port 1207, each having a respective quick disconnect 1208 and 1210. The input tube 1204 and the output tube 1206 are round tubes coupled to the input and output ports 1205 and 1207. The input and output ports 1205 and 1207 taper into a flow chamber 1212 of the treatment chamber 1202. Similar to that shown in FIG. 7C, the taper section may be designed to smoothly transition the circular fluid flow to minimize dead spots or stagnation or to achieve a substantially flat laminar fluid flow. The flow chamber 1212 extends from the input port 1205 to the output port 1207. The body portion 1214 of the treatment chamber 1202 is generally formed using multiple sheets of light transmissive material, such as described with reference to FIGS. 6 and 7A. These sheets are placed on top of each other and sealed together at the exterior edges 1216 and at the boundary 1218 to the flow chamber 1212. For example, the sheets of material are welded (e.g., radio frequency (RF) welded), or other wise bonded to each other to form the treatment chamber 1202. In some embodiments, a preform 1213 is formed in the sheets of material prior to being bonded or attached together. This preform helps that flow chamber to form as a chamber and to expand when fluid is flowed therethrough without creasing or bending along the bonded or attached portion. Thus, the treatment chamber 1202 is a generally flat and flexible structure.

The treatment chamber 1202 of FIG. 12 is similar to the treatment chamber 702 of FIGS. 7A and 7B; however, the width of the flow chamber 1212 is increased in comparison to the flow chamber 712 of FIGS. 7A and 7B. Advantageously, this allows for a greater flow rate to be obtained than with the treatment chamber 712. In one embodiment, a flow rate of 11 liters/min is obtained using the treatment chamber 1202 (in comparison to 1 liter/min with treatment chamber 702). Thus, the treatment chamber 712 is another embodiment of a flexible, flat treatment chamber that is disposable. Additionally, holes 1220 (generically referred to as alignment features) are punched into the body portion 1214 to allow for alignment within a cartridge, such as the cartridge described above. When used with a cartridge, the light transmissive plates (windows) of the cartridge restrain the flow chamber 1212 to have a substantially flat profile across the width of the flow chamber 1212 and throughout the length of the flow chamber 1212. This provides for the uniform treatment of the fluid product through all portions of the flow chamber 1212.

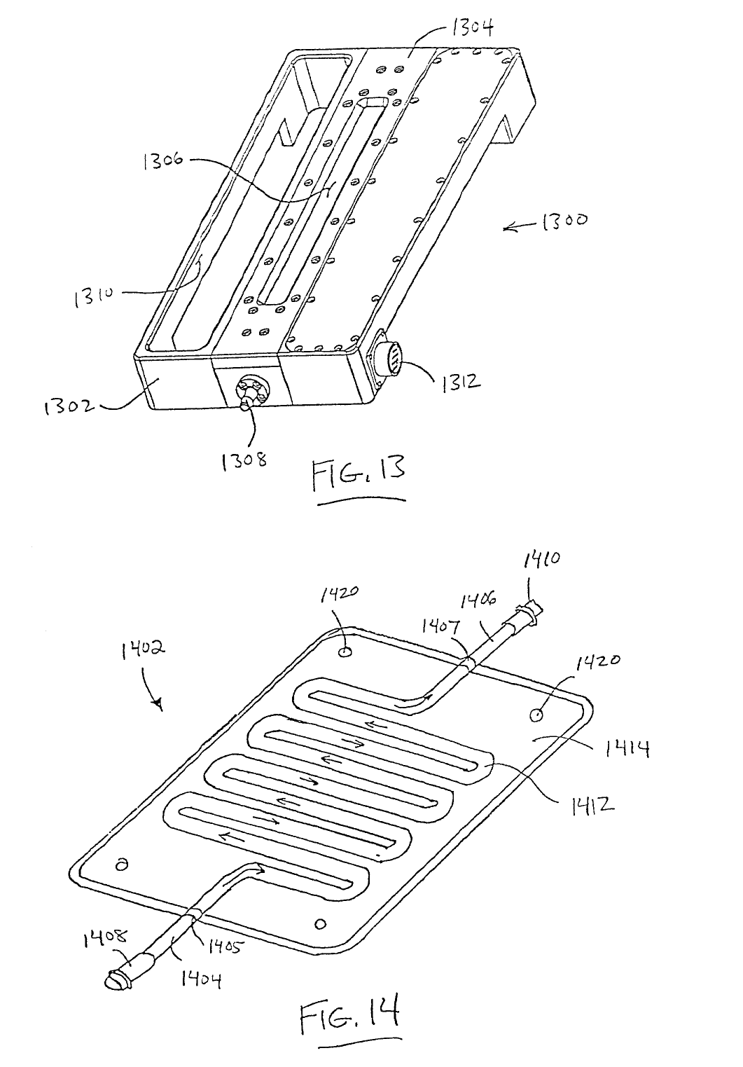

Referring next to FIG. 13, a perspective view is shown of a reusable, non-disposable treatment chamber according to another embodiment of the invention. The treatment chamber 1300 has a rigid body 1302 including a central back plate 1304 that contains a first window plate 1306. Opposite the central back plate 1304 and the first window plate 1306, is a central front plate (not shown) including a second window plate (not shown). A flow chamber (between the first and second window plates) is formed within the body portion 1302. The flow chamber may have a tubular cross section or a substantially flat cross section through the body 1302. An input port 1308 and an output port (not shown in this view) allow connection to the various flow tubes of the fluid flow path. Similar to the flexible, disposable treatment chambers of FIGS. 6-7B and 12, the reusable treatment chamber 1300 forms a flow chamber between the input port 1308 and the output port. Since the body portion 1302 is rigid, the thickness of the flow chamber can be controlled, i.e., the distance between the first and second window plates can be precisely controlled based upon the manufacturing specifications. In operation, fluid is flowed in through the input port 1308, through the flow chamber and out through the output port. As the fluid passes between the first and second window plates, the fluid is subjected to the light treatment, e.g., pulsed light treatment, to deactivate microorganisms within the fluid.

Also formed within the body portion 1302 is a handle portion 1310 to allow the operator to hold the treatment chamber 1300. It is noted that since portions of the treatment chamber 1300 are opaque, there may be a potential for slight shading to occur within portions of the flow chamber.

Additionally, in some embodiments, an electrical output 1312 is provided. Incorporated into the body portion are optional thermocouples and pressure transducers that will measure the temperature of the flow chamber and the pressure being exerted by the fluid therein, respectively. The electrical signals generated by these thermocouples and pressure transducers are output through the electrical output 1312. Thus, an electrical component adapted to mate with the electrical output 1312 transmits these signals to the system controller.

Since this treatment chamber is reusable, the treatment chamber should be cleaned and sterilized in between fluid runs. Disadvantageously, this may require disassembling the treatment chamber and cleaning it, for example, using an autoclave or other chemical flush.

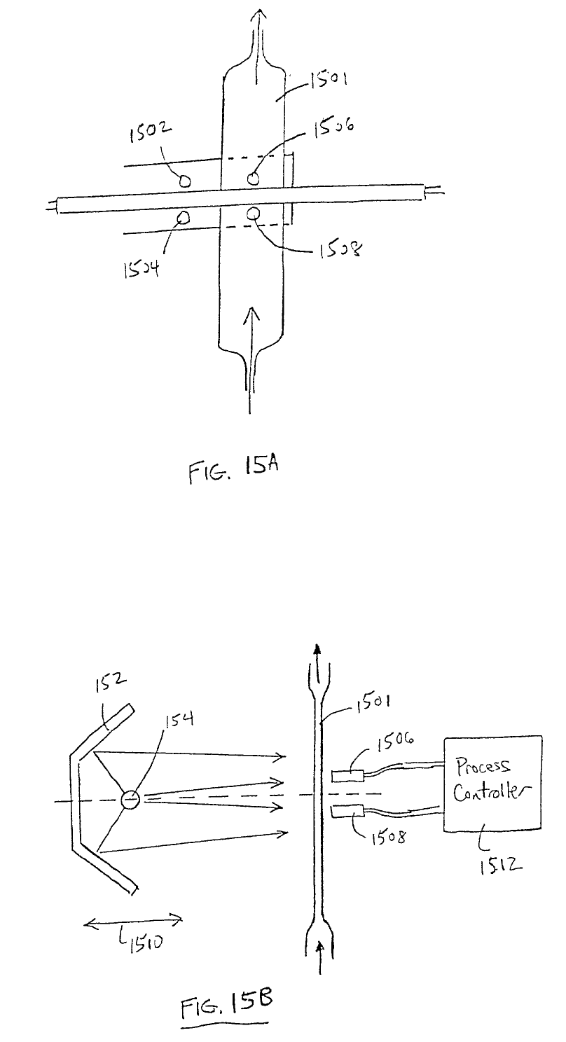

Referring next to FIG. 14, a perspective view is shown of a flat, disposable treatment chamber that may be used in the fluid treatment system of FIGS. 1-3 in accordance with another embodiment of the invention. Shown is the treatment chamber 1402 including an input tube 1404 coupled to an input port 1405, an output tube 1406 coupled to an output port 1407, each having a respective quick disconnect 1408 and 1410. A radiator flow chamber 1412 extends from the input port 1405 to the output port 1407. The radiator treatment chamber 1402 is made from light transmissive materials, such as described with reference to FIGS. 6 and 7A. The radiator patterned flow chamber 1412 is welded into the body portion 1414.

The treatment chamber 1402 of FIG. 14 is similar to the treatment chambers of FIGS. 7A, 7B, and 12; however, the flow chamber 1412 is radiator shaped such that the fluid flow path winds back and forth across the width of the treatment chamber 1402 is it progresses along the length of the treatment chamber 1402 (as illustrated by the arrows in the flow path). Advantageously, such a flow path provides for more exposure of the fluid to the pulsed light, if a similar flash rate is used. This treatment chamber 1412 is another embodiment of a flexible, flat treatment chamber that is disposable. Additionally, holes 1420 (i.e., alignment features) are punched into the body portion 1414 to allow for alignment within a cartridge, such as the cartridge described above. When used with a cartridge, the plates of the cartridge press conform the flow chamber 1412 to have a substantially flat profile across the width of the flow chamber 1412 and throughout the length of the flow chamber 1412. This provides for the substantially uniform treatment of the fluid product through all portions of the flow chamber 1412. It is noted that this is just one variation of the potential for different flow paths within the treatment chamber 1402. Depending on the duration of exposure to the light, many other flow paths could be welded into a given treatment chamber. In another embodiment, the radiator design may simply comprise a radiator shaped tubing that is rigid and is held in position in front of the lamp assembly, e.g., positioned against window 128 of FIG. 1.

It is also noted that the treatment chamber 1402 may be positioned against one or more support structures or plates that define one or more dimensional boundaries of the flow chamber 1412. Also, in embodiments constructed of sheets of flexible material bonded together, preforms may be formed in the sheets along the edges of the flow chamber to allow the flow chamber to fill with fluids without creasing or bending along the bonded locations.

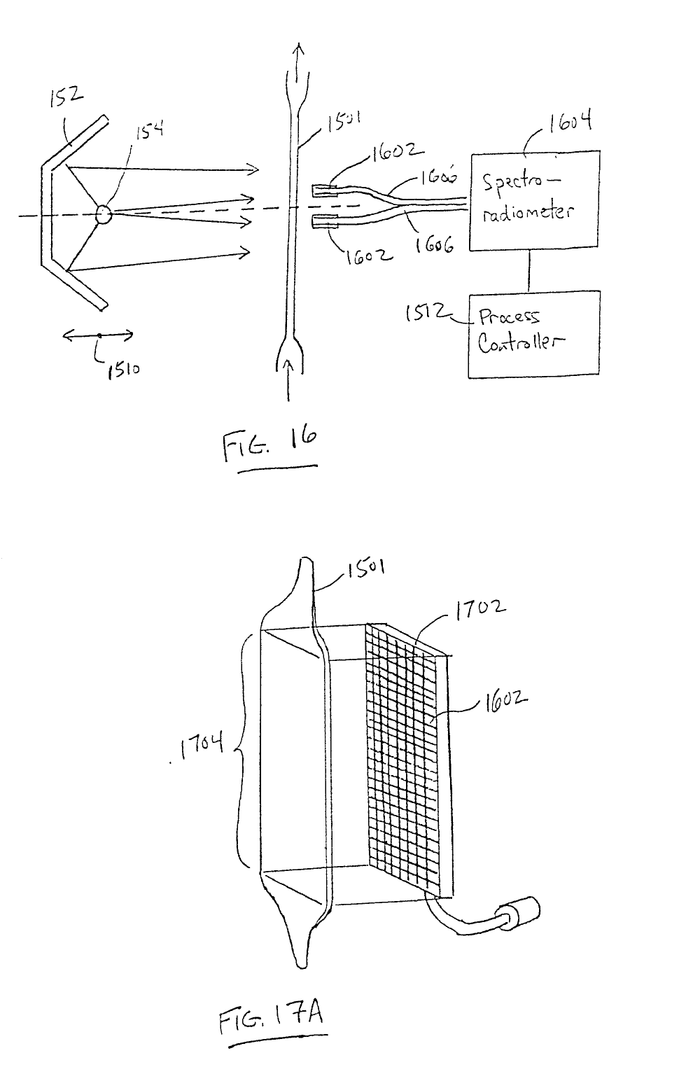

Referring next to FIG. 15A, a simplified front view is shown illustrating the relationship between the treatment chamber, the light source and the respective process monitors according to one embodiment of the invention. Concurrently referring to FIG. 15B, a simplified side view is shown of the treatment chamber, the light source and the respective process monitors. In FIG. 15A, the light source 154, e.g., flashlamp, is oriented to illuminate at least a portion of the treatment chamber 1501 (e.g., treatment chambers 610, 702, 1202, 1402). Photodetectors 1502 and 1504 (i.e., one embodiment of the process monitors 137) are positioned to view the light emitted directly from the light source 154 that reaches the treatment chamber 1501. For example, in the fluid treatment system of FIGS. 1-3, photodetectors 1502 and 1504 (i.e., one embodiment of the process monitors 139) view the light transmitting through the window 128 of the cartridge registration plate 132. Photodetectors 1506 and 1508 are positioned to view the light emitted from the light source 154 and penetrating through the treatment chamber 1501 and its fluid contents. For example, in the fluid treatment system of FIGS. 1-3, photodetectors 1506 and 1508 view the light transmitting through the window 135 of the cartridge 134 and the registration plate window 128. This allows for measurements of the fluence or intensity and the spectral content of the light reaching the treatment chamber 1501 as well as the light penetrating through the fluid product.

Additionally, since the light emitted from the light source 154 includes wavelengths from about 180 nm to 2600 nm, the photodetectors 1502 and 1506 of this embodiment are ultraviolet photodetectors or photodiodes, e.g., they measure light having wavelengths between about 230 and 400 nm. Thus, photodetectors 1502 and 1506 provide an accurate characterization of the fluence and spectral content of the UV portion of the emitted light. Furthermore, photodetectors 1504 and 1508 of this embodiment are full spectrum photodetectors are photodiodes that measure light having wavelengths between about 400 and 950 nm. Advantageously, the photodetector pairs behind the treatment chamber and to the side of the treatment chamber each include one UV photodetector and one full spectrum photodetector. It is noted that other photodetectors may be used depending on the wavelength range of the emitted light and system configuration. Thus, the photodetectors may be configured to measure light in any given range of wavelengths or of a desired single wavelength.

The photodetectors 1502 and 1504 are used to verify the fluence selected by the operator prior to operation and the fluence of each flash during operation, as well as the spectral content of the light. For example, if the operator sets the fluence level to 0.3 J/cm², before the fluid run is initiated, the power to the light source 154 is set and the light source 154 is moved in the direction of arrow 1510 such that the distance between the light source 154 and the treatment chamber 1501 is set (e.g., using the linear slide servo drive 110). The light source 154, e.g., a flashlamp, is then flashed and the fluence is measured using photodetectors 1502 and 1504. If the fluence is not at the expected level, the distance between the light source 154 and the treatment chamber 1501 is incrementally adjusted based on the pre-learned adjustments and flashed again until the photodetectors verify the selected fluence. At this point, the product run is initiated. This is an important feature when the fluid product to be treated is a blood plasma derivative or other bioprocessing media, due to the sensitive nature of the fluid product. For example, exposure to light having a high fluence level may deactivate microorganisms, but may further result in an unacceptable amount of protein damage. In some instances, such bioprocessing fluid media may be extremely expensive and/or not replaceable, such that it is important that the fluence levels are accurately set by the fluid treatment system.

It is noted that each of the process monitors may measure one or both of the fluence level of the measured light and the spectral content of the measured light. It is also noted that in some embodiments, one or more of the process monitors 1502, 1504, 1506 and 1508 may comprise an optical detector such as a photodetector, a photodiode, a fiber optic probe, a calorimeter, a joulemeter, a photomultiplier tube, a camera, and a CCD array. In other embodiments, the one or more of the process monitors 1502, 1504, 1506 and 1508 may comprise a thermodetector such as a thermocouple, a thermopile, a calorimeter, and a joulemeter.

A side view is illustrated in FIG. 15B. In this view the reflector 152 directs the light toward the treatment chamber 1501. Also seen are the UV photodetector 1506 and the full spectrum photodetector 1508. Furthermore, FIG. 15B illustrates a process controller 1512 that inputs the signals from the various process monitors and processes them to model the spectral content and/or the fluence level or intensity of the light treatment. This monitoring is used to adjust and verify the operating parameters of the fluid treatment system.

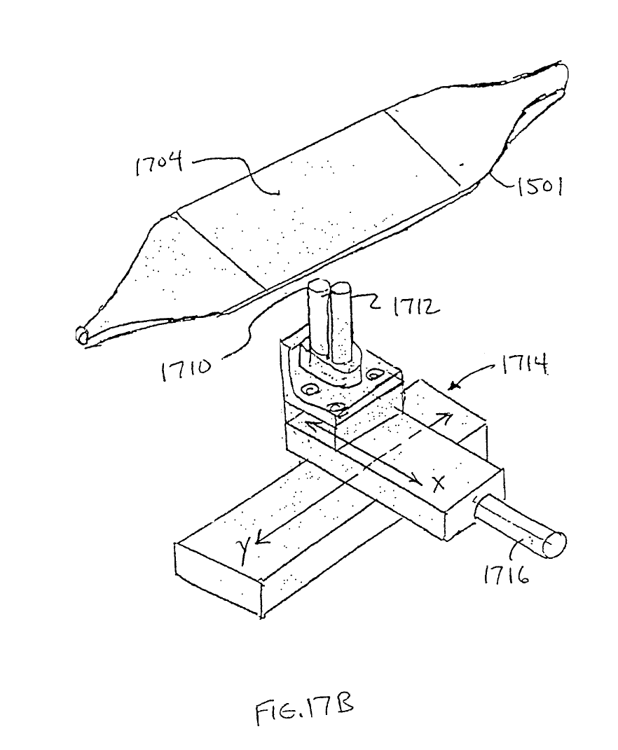

It is noted that in other embodiments, the photodetectors 1502, 1504, 1506 and 1508 may be replaced by fiber optic probes that are coupled to a spectroradiometer via fiber optic cables that measure both UV and full spectrum through the treatment chamber and directly from the light source 154, as is described with reference to FIG. 16.

It should be noted that in preferred embodiments of the invention, reflective surfaces are not employed on the through side of the treatment chamber 1501. For example, referring briefly to FIG. 8, the window 810 is light transmissive. The window 810 could be made into a reflective surface that reflects light reaching through the treatment chamber back toward the treatment chamber. However, it has been found that this additional reflected light has an effect on the fluence levels as measured by a photodetector viewing light within the chamber, i.e., the fluence level appears slightly higher than is that truly emitted from the flashlamp 154. Due to the sensitive nature of some fluid products to be treated, it is more important to obtain a consistent and accurate measurement of the fluence of the emitted light, rather than maximize the fluence within the treatment chamber. Thus, in preferred embodiments, reflective surfaces are not employed on the through side of the treatment chamber 1501.

Referring next to FIG. 16, a simplified side view is shown of a variation of the process monitoring system of FIGS. 15A and 15B according to another embodiment of the invention. According to this embodiment, rather than using discrete photodiode type photodetectors as the process monitors, fiber optic probes 1602 are provided in place of the photodetectors 1502, 1504, 1506 and 1508. Thus, the fiber optic probes 1602 are one embodiment of the process monitors 137 and 139. The output of each flash is sampled directly and through the treatment chamber 1501 via fiber optic probes 1602, which are coupled via fiber optic cables 1606 to a spectroradiometer 1604. The output of the spectroradiometer 1604 is analyzed in real time by the process controller 1512 to assure that each flash contains the proper distribution of wavelengths at the proper fluence levels or intensities, which is optimized depending on the specific pathogen or fluid product to be treated. It is noted that in embodiments using continuous wave light, the spectroradiometer is configured to process the light continuously.

The spectroradiometer 1604 is a multi-channel device including an analog to digital converter. In one embodiment, the fiber optic probes 1602 are cosine corrected irradiance probes, which are coupled to the analog to digital converter of the spectroradiometer 1604 via 200-μm fiber optic cables 1606. The spectroradiometer 1604 is integrated with software that measures the spectral intensity of each flash from the light source 154. In one embodiment, similar to that described in FIGS. 15A and 15B, two probes measure UV light (225-400 nm) and the other two measure wavelengths from 400-950 nm, one of each type of probe measuring the light directly emitted from the light source 154 and one measuring the light transmitted through the treatment chamber 1501.

In operation, whether using photodetectors 1502, 1504, 1506, 1508 or fiber optic probes 1602, prior to flowing the fluid through the treatment chamber 1501, the light source intensity is checked by flashing the light source 154. The detection system including the process controller 1512 verifies the correct spectral content and fluence or intensity. If the spectral signature is not correct, the process controller 1512 will adjust the distance of the light source 154 to the treatment chamber 1501 in order to vary the intensity over the spectral distribution prior to initiating the fluid run. Additionally, as is known, adjusting the charge voltage across the light source 154, e.g., a flashlamp, will change the spectral distribution. For example, higher charge voltages will drive the flashlamp plasma to higher temperatures and increase the UV to visible IR ratio to be delivered to the treatment chamber 1501. Thus, the use of the spectroradiometer 1604 and process controller 1512 will allow for the control and optimization of these process parameters.

Furthermore, as the fluid product is flowed or pumped through the treatment chamber, light energy absorption is calculated and monitored at various wavelengths via the fiber optic probes 1602 that view the light penetrating through the treatment chamber 1501. For example, separate curves are generated for the spectral distribution of the light emitted directly from the light source and for the light transmitting through the treatment chamber 1501. By integrating the two generated curves, two areas are obtained. By taking the difference between the two areas, the absorbed light energy is calculated at the various monitored wavelengths. This is an important metric to obtain since certain biological fluids, such as blood, blood plasma and blood plasma derivatives may incur excessive protein damage if the fluence level of the light is too high. As such, if too much energy is absorbed, there may be excessive protein damage. On the other hand, if too little energy is absorbed, pathogens and other microorganisms or contaminants may not be deactivated.

Thus, due to the sensitivity of certain bioprocessing fluids, blood plasma derivatives, etc., careful monitoring of the light treatment is needed. The use of the fiber optic probes 1602, fiber optic cable 1606 and the spectroradiometer 1604 enable accurate processing and modeling of the spectral content and intensity (fluence) of the light treatment, while the fluid treatment system provides for adjustment of the spectral content and intensity of the light treatment in response to processing the light treatment.

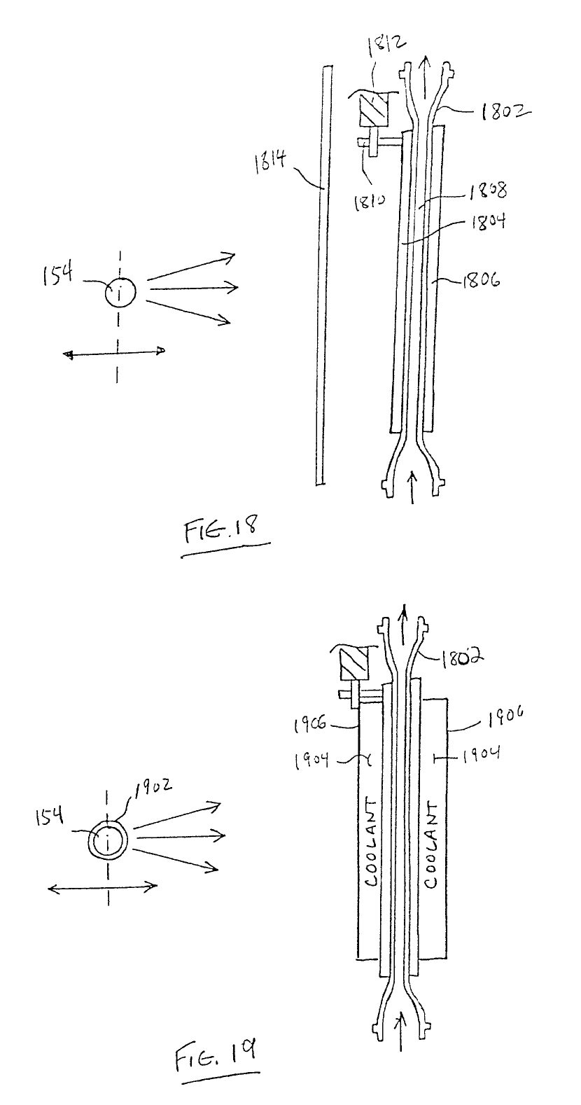

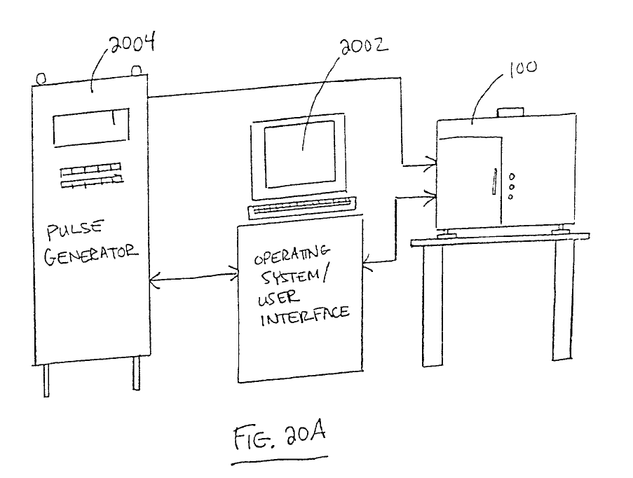

Referring next to FIG. 17A, a simplified perspective view is shown of detector array that is used to obtain the spectral profile of the light treatment across the entire treatment chamber according to yet another embodiment of the invention. Illustrated is the treatment chamber 1501, which may be any of the treatment chambers described herein. Rather than two process monitors to measure the light transmitted through the treatment chamber, e.g., photodetectors or fiber optic probes, a detector array 1702 is positioned behind the entire treatment zone 1704 (which corresponds to the profile of the flow chamber of treatment chamber 1501 in this embodiment) of the treatment chamber 1501. For example, the detector array 1702 is an array of fiber optic probes 1602 arranged in a grid behind the treatment zone 1704. In alternate embodiments, the fiber optic probes 1602 may be discrete photosensitive devices (e.g., photodiodes) or may comprise a charged coupled device (CCD) array.

The use of the detector array 1702 provides the process controller with the measurements to create a dose mapping of a profile of the treatment zone 1704 (e.g., a profile of the flow chamber) of the treatment chamber 1510. Thus, light energy transmitting through treatment chamber 1501 is collected across the entire treatment zone 1704 (or scan area). With no fluid flowing, this detector array 1702 will test the uniformity of the light treatment across the entire treatment zone 1704. The same can be tested by pumping a fluid having a known consistent optical density, such as water or another more absorbing fluid. In operation, with the fluid product being pumped through the treatment chamber 1501, the uniformity of the absorption of the light treatment of the fluid is tested. As described, above, particularly with blood plasma derivatives and other bioprocessing fluids, it is important to obtain uniform treatment of the fluid product so that excessive protein damage is prevented while at the same time maximizing the effective kill rate of pathogens, bacteria and other microorganisms.

It is noted that in some embodiments the detector array 1702 may be positioned to measure light passing through at least a portion of the profile of the treatment zone 1704. For example, the detector array structure may be sized smaller than the profile of the treatment zone 1704 or the fiber optic probes 1602 (or other optical detectors) may only cover a portion of the detector array structure. In such embodiments, the detector array 1702 may be sized to measure the light penetrating through less than the entire portion of the flow chamber or treatment zone 1704. Thus, the detector array creates a dose mapping of at least a portion of the profile of the treatment zone 1704.

In some embodiments, a lens system (not shown) may be positioned between the process monitors, e.g., photodetectors 1506 and 1508 or fiber probes 1602, and the treatment chamber to focus the transmitted light into the respective process monitor. Such a lens system could comprise a single lens or multiple lenses. Thus, a lens may be positioned in between each process monitor and the treatment chamber. In other embodiments, a lens may be positioned in between the treatment chamber and a CCD array (not shown) in order to focus the energy of the emitted light into the CCD array. In these embodiments, the CCD array is another alternative type of process monitor.

Referring next to FIG. 17B, a simplified perspective view is shown of process monitors integrated on an adjustable x-y translation table used to obtain the spectral profile of the light treatment across different portions of the treatment chamber according to yet another embodiment of the invention. In this embodiment, process monitors 1710 and 1712 are integrated into an x-y translation table 1714, which adjusts the position of the x-y position of the respective process monitors 1710 and 1712 under the treatment zone 1704 of the treatment chamber 1501. Such x-y translation tables 1714 are well known in the art. The output 1716 allows the process monitor outputs to be coupled to a process controller. These process monitors 1710 and 1712 may be fiber optic probes, photodiodes (or other photodetectors), pressure transducers or thermopiles. In one embodiment, process monitor 1712 is a fiber optic probe (or alternatively, a photodetector) that is configured to measure UV light, e.g., 225-400 nm, while process monitor 1710 is a fiber optic probe (alternatively, photodetector) that is configured to measure light from 400-950 nm. It is noted that these process monitors may be configured to measure light having any specified range of wavelengths or a single wavelength.

The process monitors 1710 and 1712 are mounted to continuously scan at least a portion of the treatment zone 1704, e.g., the entire treatment zone 1704, for calibration and/or process monitoring during a fluid run. This would provide additional information relating to the uniformity of the light treatment across the treatment area and may identify areas of fouling and identify areas not being adequately treated.

Referring next to FIG. 18, a simplified side view is shown of a treatment chamber including a spectral filter positioned between the treatment chamber and the flashlamp according to another embodiment of the invention. Illustrated is the treatment chamber 1802 held in between a first window plate 1804 and a second window plate 1806 defining a thickness of a flow chamber of the treatment chamber 1808 (i.e., defining two dimensional boundaries of the flow chamber). The thickness of the flow chamber is adjustable by adjusting a screw 1810 of a cartridge 1812 (or digital precision spacers or other spacing structure, e.g., spacers 814 of FIG. 8). In order to filter portions of the emitted light from the flashlamp 154, a filter 1814 is positioned in between the flashlamp 154 and the treatment chamber 1802. The filter may be positioned in a variety of ways. For example, referring to FIGS. 1-3, the filter 1814 may be positioned on either side of window 128, or may be positioned within the cartridge 134. For example, referring to FIG. 8, the filter 1814 may be positioned in between the cartridge top 802 and the first window 806. Advantageously, this filter 1814 allows for the selectable spectral filtering of the light from the light source 154. It is noted that the structure that defines the distance between the two windows or plates is positioned outside of the two plates in some embodiments (as shown); however, may be positioned in between the two plates in other embodiments (e.g., a spacer held in position in between first window plate 1804 and a second window plate 1806).

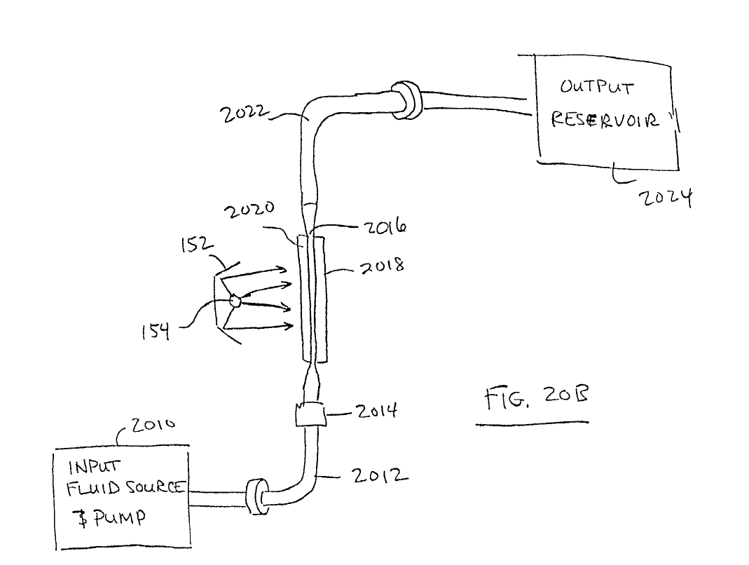

Referring next to FIG. 19, a simplified side view is shown of a treatment chamber including a device to cool the treatment chamber due to the heat energy of the light illuminating the treatment chamber according to another embodiment of the invention. Although the usage of Xenon flashlamps generates a considerable amount of heat, in many embodiments, means to cool the treatment chamber and the light source 154 is not provided. This is due to relatively short period of time of operation in the completion of a single fluid run. Generally, the treatment chamber does not heat up enough to affect the fluid product.

However, a production scaled version of the fluid treatment system may operate for several hours continuously. Thus, in such systems, a means to cool the treatment chamber is provided. Additionally, the light source 154 itself may be cooled, for example, by pumping water or another liquid through a sheath 1902 surrounding the light source 154. Likewise, the treatment chamber may be cooled by flowing a cooling medium 1904, such as water or air, through a conduit 1906 or sheet positioned against the transmissive windows holding the treatment chamber 1802. In other embodiments, the cooling is provided by a chill plate, heat exchanger or vortex coolers, or even immersing treatment chamber into a bath of cooling material. Alternatively, cooling tubes could be adhered to the exterior of the windows holding the treatment chamber 1802.

Referring next to FIG. 20A, a system level diagram is shown for a fluid treatment system according to one embodiment of the invention. Illustrated are the fluid treatment system 100, the computer operating system/user interface 2002 and the pulse generator 2004. The computer operating system /user interface 2002 includes the main processing and control software to operate the fluid treatment system 100. The user is able to set the specific parameters for the fluid treatment device for its operation, e.g., the pump rate, the spectral distribution of the light, the fluence or intensity of the light, number of flashes, etc. The operating system /user interface 2002 also receives feedback and monitoring signaling from the fluid treatment system 100 as well as controls the pulse generator 2004. The pulse generator 2004 generates the pulses to be delivered to the fluid treatment system 100 and is produced as PUREBRIGHT Model No. PBS-1 available from PurePulse Technologies, Inc. of San Diego, Calif., USA. The pulse generator 2004 includes a pulsing device that includes a DC power supply that charges energy storage capacitors; a switch used to discharge the capacitors; a trigger circuit used to fire the switch at pre-programmed time intervals; and a set of high voltage coaxial cables carrying the discharge pulses from a capacitor-switch assembly to the flashlamp within the housing fluid treatment system 100.

Referring next to FIG. 20B, a simplified schematic drawing is shown of production fluid treatment system scaled to continuously treat fluids. A constant fluid source 2010 is coupled to an input tube 2012 (supply conduit). The constant fluid source 2010 may be a large fluid reservoir or container having a pump or pumping mechanism to provide the fluid flow at a specified rate. A flow rate detector 2014 may be incorporated into tube 2012 to detect the rate of the fluid flow. The detected rate may be used to set the flash rate of the light source 154 (in pulsed light embodiments). The fluid flows through the treatment chamber 2016, which may be similar to those described throughout this specification. The treatment chamber is positioned between two light transmissive support structures (plates or windows 2018 and 2018). These structures define at least one dimensional boundary of the treatment chamber 2016. These structures may be integrated into a cartridge as described above or be separate structures hinged to hold the treatment chamber in position. Additionally, the distance between the plates may be made variable using spacers or spacing structures, for example. Furthermore, in some embodiments, these plates may be used to conform the flow chamber of the treatment chamber to a substantially uniform flow geometry. The treated fluid continues to flow out of the treatment chamber through output tube 2022 and into the output reservoir 2024. This embodiment may require cooling of the treatment chamber depending on the fluence of the flashlamp 154 and the number of flashes, for example. As such, cooling mediums, such as water or air could be circulated over the light transmissive plates 2018 and 2020.

It is noted that in alternative embodiments, the treatment chamber 2016 is not positioned between structures or plates, it is simply positioned to receive light from the light source 154. The treatment chamber 2016 may be flexible or rigid and is preferably removable and disposable. The flow through the treatment chamber 2016 may be any geometry and may provide a flat flow, a laminar flow, a tubular flow, a uniform flow, or a turbulent flow, for example, or any flow as dictated by the dimensions of the treatment chamber (or as dictated by the structures 2018 and 2020 restraining the treatment chamber 2016 and defining at least one dimensional boundary of the treatment chamber 2016). Advantageously, since the treatment chamber is disposable, it may be replaced periodically, rather than having to clean or sterilize it.

Next, the following examples are experimental results using a device similar to the fluid treatment system of FIG. 11 to illustrate the response of proteins in blood plasma derivatives to pulsed light, e.g., BSPL treatment emitted from the light source, as well as the deactivation of microorganisms, such as E. coli.

### EXAMPLE 1

### Protein Damage

Various proteins, such as Alkaline Phosphatase, Lactate Dehydrogenase, acid Phosphatase and Beta Galactoidase were tested for their susceptibility to BSPL. Each protein was contained within fluid at a total protein concentration of 5 mg/ml and treated in a static chamber. Treatments were formed in 1 ml samples, in replicates of three. Each sample was subjected to N 0.25 J flashes of BSPL, where N=1, 2, 4, 6, 8, and 12. This corresponds to a total energy of 0.25 J, 0.5 J, 1.0 J, 1.5 J, 2.0 J and 3.0 J, respectively for N=1, 2, 4, 6, 8 and 12. Following the treatment, each protein or enzyme was assayed to determine the percent of enzyme activity remaining. The result is plotted in FIG. 21 as a % of protein activity remaining vs. the number of flashes.

As seen in FIG. 21, different proteins (enzymes) are susceptible to BSPL to differing extents. Line 2102 corresponds to Alkaline phosphate, line 2104 corresponds to Lactate Dehydrogenate, line 2106 corresponds to Acid Phosphatase, and line 2108 corresponds to Beta galactosidase. Alkaline phosphatase is very resistant to BSPL showing no loss of protein activity even with 3 joules of total energy, whereas beta-galactosidase is far less resistant showing activity loss with as little as 0.25 joules of total BSPL energy. Thus, since it is desired to deactivate pathogens within the bioprocessing fluids with minimal protein damage, the fluence of the fluid treatment and the number flashes that portions of the fluid are subjected to will vary greatly depending on the specific proteins present in the bioprocessing fluid.

### EXAMPLE 2

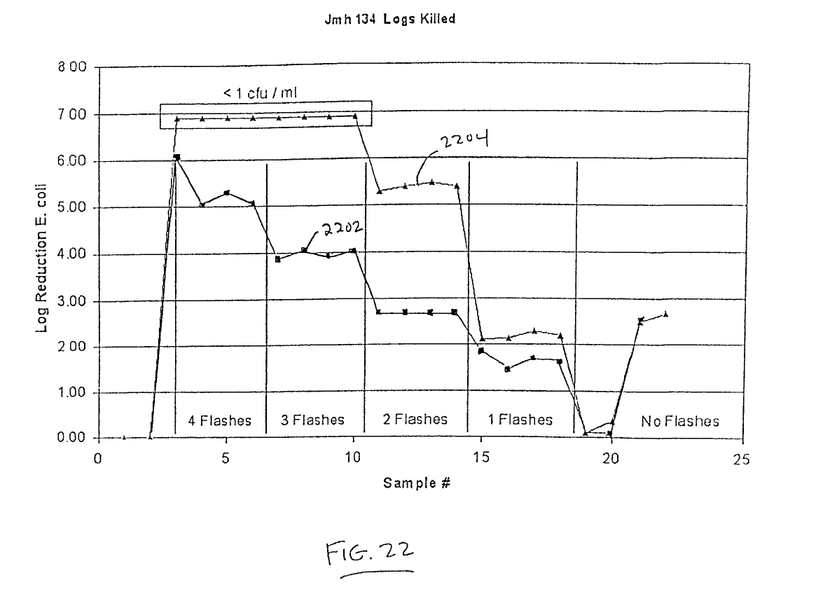

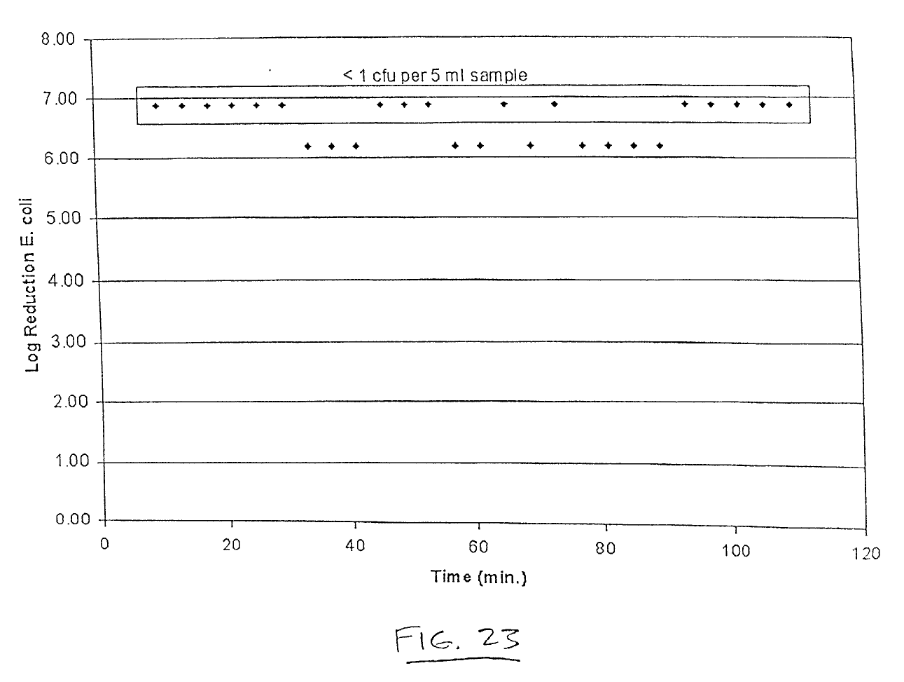

Example 2 involves the use of the “Staircase” test to determine treatment kinetics and system response in-flow of the fluid treatment system of FIG. 10 with fluid containing 5 mg/ml bovine serum albumin (BSA). BSA is a form of serum albumin that is a known protein that is effective in protecting other molecules from degradation due to BSPL. BSA is a readily available source of serum albumin, which is commonly used in in vitro biological studies, as a replacement for human albumin. The samples were pumped at a flow rate of 250 ml/min. The experiment provides a high initial treatment level, gradually decreasing to no treatment over the course of a 20-minute test run. The flash rate of the pulse generator coupled to the flashlamp is synchronized with the fluid flow rate to provide an effective treatment of 4, 3, 2, 1 and 0 pulses. The sample rate is 1 sample per minute. The fluid sample was also inoculated with E. coli. The results are plotted in FIG. 22 for two different treatment levels, a “low” fluence of 0.1 J/cm² per flash and a “high” fluence of 0.2 J/cm² per flash. As seen in FIG. 22, line 2202 represents the low fluence while line 2204 represents the high fluence. This data shows how parameters such as flash rate, flow rate, number of flashes and fluence per flash can be tuned to provide the desired level of microbial kill and/or product activity recovery.

### EXAMPLE 3

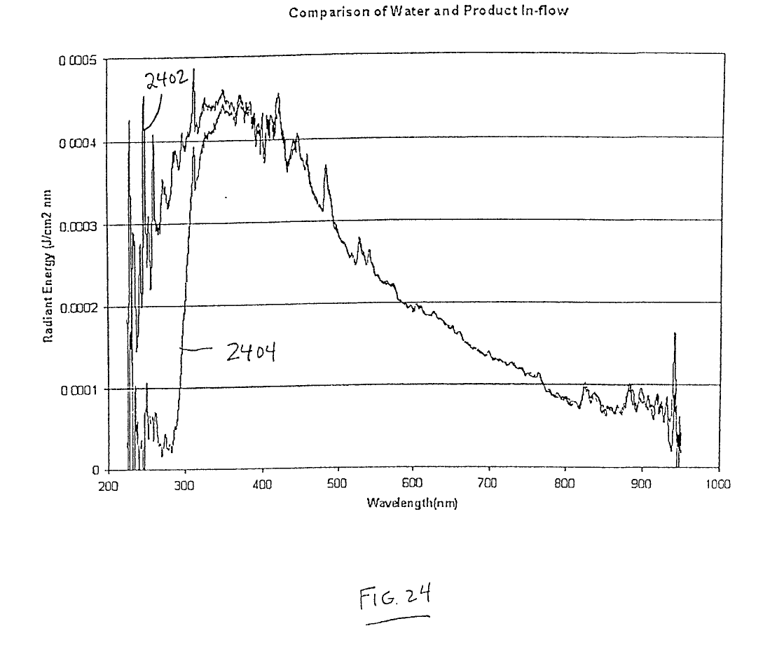

Based on optimization tests performed, such as in EXAMPLE 2, an optimum operating point was selected to provide a desired kill level of E. coli and operated for approximately two hours. In this example, the protein concentration (BSA) was 5 mg/ml BSA and the flow rate was 300 ml/min. The fluid also contained E. coli and Beta galactosidase. The treatment level was 0.5 J/cm² per flash and the total energy was between 1.5 and 3 J/cm². Samples were taken every 4 minutes in the extended run and the results of the log reduction of E. coli vs. the time the sample are plotted in FIG. 23. As can bee seen in FIG. 23, over the two-hour period, the level of kill was between 6 and 7 logs reduction, i.e., a most desirable range for pathogen inactivation and a commonly accepted level of sterilization for many applications. It is noted that an alternate pump assembly and fluid container was used to allow the test fluid to be pumped continuously for two hours (as opposed to the syringes described above). Thus, as can be seen, BSPL is very effective in deactivating pathogens, even while operated under parameters to minimize protein damage in bioprocessing fluids, such as blood plasma derivatives.

### EXAMPLE 4

### Treatment Depth 3 mm

In EXAMPLES 4-6, tests were performed to test both kill (in these experiments E. coli) and protein activity degradation (in this case Beta galactosidase or Beta gal.) as experimental outputs. In many embodiments, it is a goal to achieve a high level of kill to a low level of protein activity degradation or protein damage. Thus, a useful metric for an indication of treatment efficacy and as a tool for treatment optimization is the ratio of protein damage (in % activity reduction) to the kill level (in logs reduction). A lower damage/kill ratio is better. For example, 5 logs of kill with 30% Beta-gal. damage provides a damage/kill ratio=6. Five logs kill with 25% damage provides a better damage/kill=5.

In EXAMPLE 4, Bovine serum albumin (BSA) (Sigma 40K0898) was reconstituted to concentrations of (5, 10, 15, 25 and 50) mg/ml, mixed with 3 mg/ml Beta-galactosidase (ICN 7026B) at a 1 to 1000 dilution (Beta-galactosidase activity is used to monitor protein damage) and inoculated with E. coli (ATCC 11775) to 106 cfu/ml. Each inoculated concentration was pumped through a 1/11th-laboratory scale treatment chamber (e.g., treatment chamber 702) at a flow rate of 200 ml/min with a treatment depth of 3 mm (as adjusted by altering the distance between the respective window plates of a cartridge). As the concentrations of the fluid passed through the treatment chamber each was exposed to broad spectrum pulsed light from a single flashlamp positioned to deliver energy levels between 0.1 J/cm² and 0.68 J/cm² per flash. The flash frequency was varied based on the center line velocity such that the fluid passing through the center of the treatment zone received between 1 and 5 exposures. Samples of treated concentrations were collected and assayed for Beta-galactosidase activity and E.coli kill.

The results of these tests are shown in TABLE 1. The number in TABLE 1 is the protein damage to kill ratio and the number in parenthesis is the number of flashes needed. E.coli kill was found to be BSA concentration dependent through all energy levels tested. Greater than 6 logs of kill was achieved at fluence or energy levels of (0.2, 0.3, 0.4, and 0.68) J/cm2 per flash for differing concentrations of BSA. The respective concentration and number of flashes at each of these flowing conditions was (4 flashes at 0.2 J/flash for 5 mg/ml BSA), (5 flashes at 0.3 J/flash for 10 mg/ml BSA), (3 flashes at 0.4 J/flash for 10 mg/ml BSA) and (4 flashes at 0.68 J/flash for 15 mg/ml BSA). Protein damage measured as a function of Beta-galactosidase activity for each of the above flowing conditions was less than 30% in all cases. This corresponds to damage/kill ratios of 6 or less. For example, in some cases, the damage to kill ratio is less than 5, less than 4, less than 3, and less than 2.

TABLE 1
Protein Damage/Kill ratio (Treatment Depth 3 mm)

| [BSA] | | | | | |
|---|---|---|---|---|---|
| (mg/ml) | 0.1 J/flash | 0.2 J/flash | 0.3 J/flash | 0.4 J/flash | 0.68 J/flash |
| 0 | 1.0 (2)* | 5.0 (1) | 6.0 (1) | 9.0 (1) | 15.8 (1) |
| 5 | | 4.2 (4) | 2.9 (4)* | 3.8 (3) | 6.1 (2) |
| 10 | | | 5.0 (9)** | 3.3 (3-4)* | 4.4 (3) |
| 15 | | | | 5.0 (8)** | 4.3 (4-5)* |
| 25 | | | | | 9.0 (15)** |
| 50 | | | | | >12 (25)** |

*optimum test condition for a given concentration of BSA
**extrapolated value

### EXAMPLE 5

### Treatment Depth 1 mm

In this example, Bovine serum albumin (BSA) (Sigma 40K0898) was reconstituted to concentrations of (5, 10, 15, 25 and 50) mg/ml, mixed with 3 mg/ml Beta-galactosidase (ICN 7026B) at a 1 to 1000 dilution (Beta-galactosidase activity is used to monitor protein damage) and inoculated with E.coli (ATCC 11775) to 10⁶ cfu/ml. Each inoculated concentration was pumped through a 1/11th-laboratory scale treatment chamber (e.g., treatment chamber 702) at a flow rate of 61 ml/min with a treatment depth of 1 mm. As the concentrations passed through the treatment chamber each was exposed to board spectrum pulsed light from a single lamp positioned to deliver fluence or energy levels between 0.1 J/cm² and 0.3 J/cm² per flash. The flash frequency was varied based on the center line velocity such that the fluid passing through the center of the treatment zone received between 1 and 5 exposures. Samples of treated concentrations were collected and assayed for Beta-galactosidase activity and E.coli kill.

The results of these tests are shown in TABLE 2. Again, E.coli kill was found to be BSA concentration dependent through all energy levels tested. Greater than 5 logs of kill was achieved at different fluence or energy levels (of 0.1, 0.2 and 0.3 J/cm2 per flash) at differing concentrations of BSA. Respective concentration and number of flashes at each of these flowing conditions was (4 flashes at 0.1 J/flash for 25 mg/ml BSA), (3 flashes at 0. 2 J/flash for 25 mg/ml BSA) and (5 flashes at 0. 3 J/flash for 50 mg/ml BSA). Protein damage measured as a function of Beta-galactosidase activity for each of the above flowing conditions was less than 25% in all cases. Thus, as can be seen damage/kill ratios of less than 5, less than 6, less than 4, and less than 3 are achievable, respectively.

TABLE 2
Protein Damage/Kill ratio (Treatment Depth 1 mm)

| [BSA] (mg/ml) | 0.1 J/flash | 0.2 J/flash | 0.3 J/flash |
|---|---|---|---|
| 0 | 12.8 (1) | 13.6 (1) | |
| 5 | 6.0 (2-3) | 6.7 (2) | |
| 10 | 2.5 (3-4)* | 5.2 (2-3) | |
| 15 | 3.1 (4-5)* | | |
| 25 | 3.6 (5-6)* | 4.0 (4) | 3.9 (4) |
| 50 | | | 4.4 (5-6)* |

*optimum test condition for a given concentration of BSA

### EXAMPLE 6

### Treatment Depth 0.2 mm

In this example, Bovine serum albumin (BSA) (Sigma 40K0898) was reconstituted to concentration of 100 mg/ml, mixed with 3 mg/ml Beta-galactosidase (ICN 7026B) at a 1 to 1000 dilution (Beta-galactosidase activity is used to monitor protein damage) and inoculated with E.coli (ATCC 11775) to 10⁶ cfu/ml. The inoculated concentration was pumped through a 1/11th-laboratory scale treatment chamber at a flow rate of 20 ml/min with a treatment depth of 0.2 mm. As the solution passed through the treatment chamber it was exposed to board spectrum pulsed light from a single lamp positioned to deliver a fluence or energy level of 0.1 J/cm² per flash. The flash frequency was varied based on the center line velocity such that the fluid passing through the center of the treatment zone received between 1 and 5 exposures. Samples of treated concentrations were collected and assayed for Beta-galactosidase activity and E.coli kill.

A protein damage/kill ratio of 5.2 was obtained with a 100 mg/ml concentration of BSA treated with 0.1 J/flash, and yielding greater than 1.5 logs of kill (e.g., 1.7). The respective number of flashes at this flowing condition was 4 flashes. Protein damage measured as a function of Beta-galactosidase activity for the above flowing condition was less than 10%.

### EXAMPLE 7

### Spectral Profile

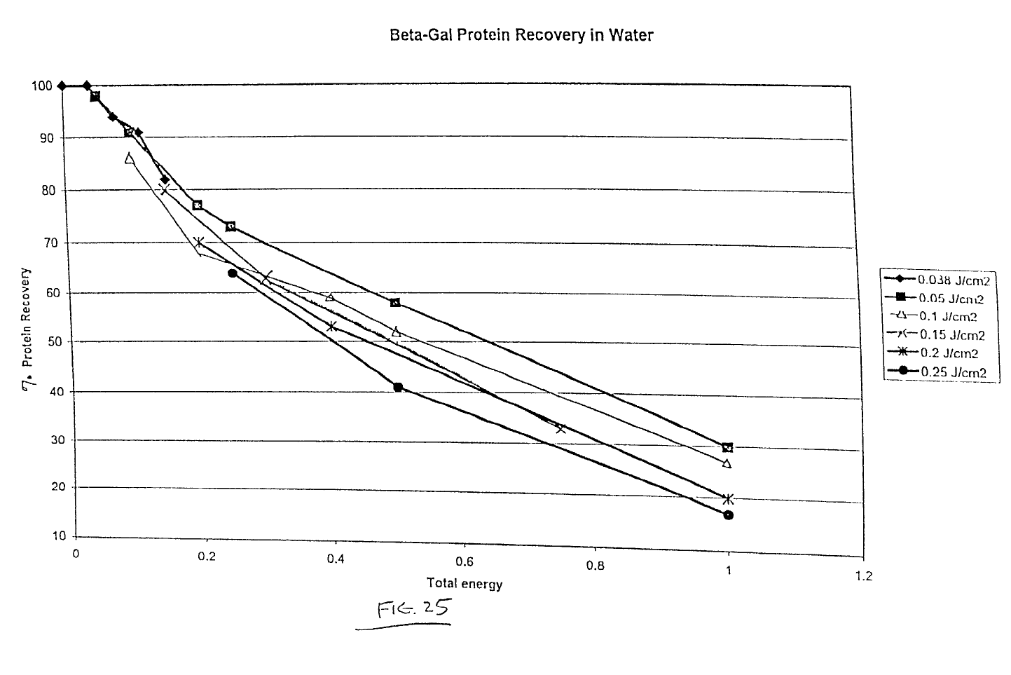

In this example, and referring to FIG. 24, an illustration is shown of the output of spectral irradiance monitoring instrument (SIMI) in monitoring light transmitted through the treatment chamber during a product run. Water is initially pumped through the treatment chamber to establish flow within the system and provide baseline diagnostic data. The curve 2402 shows a typical spectral radiant energy measurement when water is flowing through the treatment chamber, compared to the spectral radiant energy measured through a protein solution product as shown in curve 2404. Note that the measurements are nearly identical for wavelengths above 400 n. This sample protein solution absorbs significantly below 400=n, causing significantly lower UV energy measurement compared to the water. The ratio of the two measurements as well as the spectral signature of the protein solution can be very useful in analyzing the characteristics of the protein solution and the parameters of the treatment. It is noted that the difference between the two curves 2402 and 2404 at a given wavelength represents the amount of radiant energy absorbed by the protein solution at the given wavelength.

### EXAMPLE 8

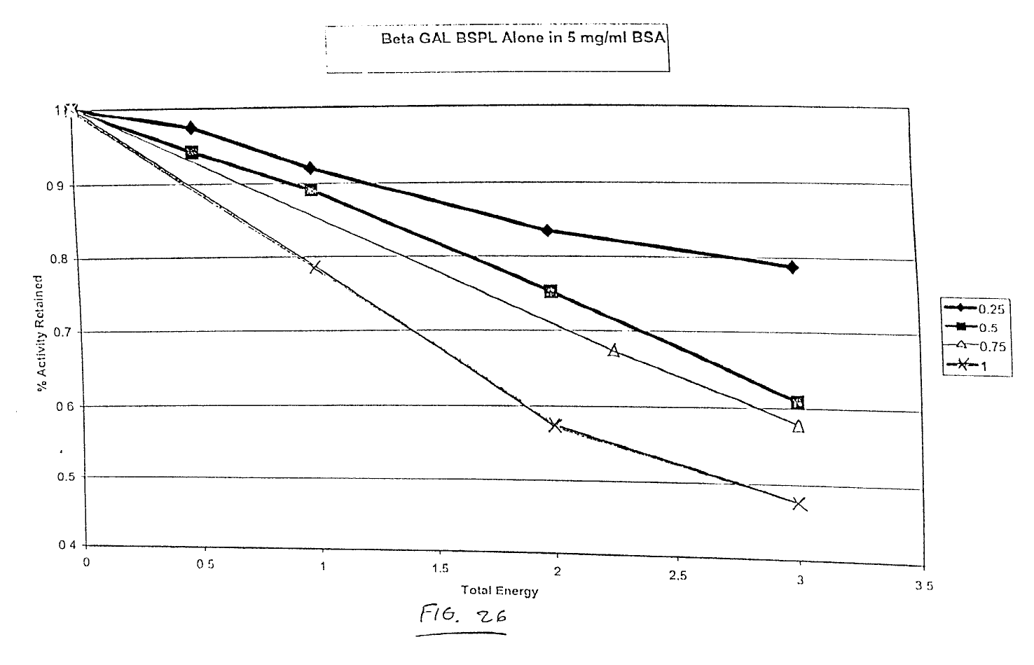

As illustrated in FIG. 25, a graph is shown of percentage of protein recovery vs. the total energy of BSPL for various fluence levels /flash. In this case, the protein tested was Beta-galactosidase within water at flashes of 0.038, 0.05, 0.1, 0.15, 0.2, and 0.25 J/flash. It can be seen that generally at lower fluence levels, such as 0.038 J/cm² and 0.05J/cm², more protein activity of the Beta-gal. remains after light treatment.

### EXAMPLE 9

As illustrated in FIG. 26, the percentage of protein activity remaining of Beta-gal within 5 mg/ml BSA vs the total energy of light illuminating the solution is illustrated. The solution was tested with flash fluence or intensities of 0.25, 0.5, 0.75 and 1 J/cm². Again, as seen, at lower fluence levels, such as 0.25 and 0.5 J/cm², the percentage of remaining protein activity is highest.

Other examples and test results involving the illumination of biological fluids, such as blood plasma derivatives with pulsed polychromatic light, such as BSPL, are provided variously in the following co-pending patent applications, each of which is incorporated herein in its entirety by reference: U.S. application Ser. No. 09/329,018, to Cover et al., filed Jun. 9, 1999, entitled METHODS OF INACTIVATING VIRUSES, BACTERIA AND OTHER PATHOGENS, IN BIOLOGICALLY DERIVED COMPOSITIONS, USING BROAD-SPECTRUM PULSED LIGHT; U.S. application Ser. No. 09/502,190, to Cover et al., filed Feb. 11, 2000, entitled PROTECTING MOLECULES IN BIOLOGICALLY DERIVED COMPOSITIONS WHILE TREATING WITH BROAD-SPECTRUM PULSED LIGHT; and U.S. application Ser. No. 09/596,987, to Holloway et al., filed Jun. 20, 2000, entitled THE INACTIVATION OF NUCLEIC ACIDS USING BROAD-SPECTRUM PULSED LIGHT, all of which are incorporated herein by reference.

While the invention herein disclosed has been described by means of specific embodiments and applications thereof, numerous modifications and variations could be made thereto by those skilled in the art without departing from the scope of the invention set forth in the claims.

What is claimed is:

1. A fluid treatment system comprising:
a light source for providing light; and
a flexible treatment chamber having an input port and an output port, at least a portion of the flexible treatment chamber positioned to receive the light;
the at least the portion of the flexible treatment chamber transmissive to at least 1% of the light having at least one wavelength within a range of 170 to 2600 nm;
the flexible treatment chamber adapted to allow a fluid to be treated to be flowed via the input port therethrough at a specified rate and out the output port,
wherein the light source illuminates the fluid as it flows through the flexible treatment chamber in order to deactivate pathogens within the fluid.

2. The system of claim 1 wherein the flexible treatment chamber is made of a light transmissive plastic material.

3. The system of claim 1 further comprising a light transmissive window that supports the flexible treatment chamber and defines at least one dimensional boundary of a treatment zone of the flexible treatment chamber.

4. The system of claim 1 further comprising a first support structure positioned in between the light source and the flexible treatment chamber, the first support structure transmissive to at least a portion of the light.

5. The system of claim 4 further comprising a second support structure positioned to hold the flexible treatment chamber in between the second support structure and the first support structure.

6. The system of claim 1 further comprising a rigid cartridge for holding the flexible treatment chamber in position and defining at least one dimensional boundary of a treatment zone of the flexible treatment chamber.

7. The system of claim 6 further comprising a cartridge registration plate, wherein the rigid cartridge is held in position by the cartridge registration plate.

8. The system of claim 1 further comprising a first support structure and a second support structure restraining the flexible treatment chamber therebetween.

9. The system of claim 8 wherein the distance between the first support structure and the second support structure is adjustable.

10. The system of claim 8 further comprising a spacer positioned between the first support structure and the second support structure defining a distance between the first support structure and the second support structure.

11. The system of claim 1 further comprising a first process monitor for measuring the light emitted directly from the light source.

12. The system of claim 11 further comprising a second process monitor positioned such that at least a portion of the treatment chamber is in between the second process monitor and the light source, the second process monitor for measuring light penetrating through the flexible treatment chamber.

13. The system of claim 12 wherein one or more of the first process monitor and the second process monitor comprise an optical detector selected from a group consisting of: a photodetector, a photodiode, a fiber optic probe, a calorimeter, a joulemeter, a photomultiplier tube, a camera, and a CCD array.

14. The system of claim 12 wherein one or more of the first process monitor and the second process monitor comprise a thermodetector selected from a group consisting of: a thermocouple, a thermopile, a calorimeter, and a joulemeter.

15. The system of claim 1 wherein the light source comprises a pulsed light source that provides pulses of light.

16. The system of claim 1 further comprising an actuator assembly coupled to the flexible treatment chamber for flowing the fluid through the flexible treatment chamber at the specified flow rate.

17. The system of claim 1 further comprising a fluid container containing the fluid to be treated and coupled to the flexible treatment chamber.

18. The system of claim 1 wherein the flexible treatment chamber comprises a bag-like structure, the fluid to be flowed therethrough.

19. A light treatment system for treating fluid products comprising:
a light source for providing light;
a treatment chamber positioned to receive the light and for allowing the fluid products to be flowed therethrough; and
a support structure supporting the treatment chamber and defining at least one dimensional boundary of a treatment zone of the treatment chamber, at least a portion of the treatment chamber and at least a portion of the support structure being transmissive to at least 1% of the light having at least one wavelength within a range of 170 to 2600 nm;
wherein the light source illuminates the fluid products as they flow through the treatment chamber in order to deactivate pathogens within the fluid products.

20. The system of claim 19 wherein the support structure comprises:
a light transmissive window positioned between the light source and the treatment chamber; and
a plate positioned to support the treatment chamber between the plate and the light transmissive window.

21. The system of claim 20 wherein the light transmissive window comprises a flat light transmissive window and the plate comprises a flat plate.

22. The system of claim 19 further comprising a flow chamber formed within the treatment chamber, the fluid products flowing through the flow chamber, wherein the support structure restrains the flow chamber to define the at least one dimensional boundary of the treatment zone while the fluid products are flowed therethrough.

23. The system of claim 22 wherein the flow chamber substantially conforms to the dimensions of at least one side of the volume created for the treatment chamber by the support structure.

24. The system of claim 22 further comprising means to control the restrainment of the flow chamber in order to control the thickness of the treatment zone.

25. The system of claim 19 further comprising at least one spacer coupled to the support structure for defining a thickness of the treatment zone.

26. The system of claim 19 wherein the support structure comprises a cartridge, the treatment chamber positioned within the cartridge.

27. The system of claim 19 wherein the treatment chamber comprises a flexible treatment chamber.

28. A disposable light treatment chamber comprising:
a flexible flow chamber transmissive to at least 1% of a light treatment having at least one wavelength within a range of 170 to 2600 nm, the flexible flow chamber adapted to allow a fluid to be flowed therethrough and illuminated with the light treatment such that pathogens within the fluid are deactivated by the light treatment;
an input port formed at one part of the flexible flow chamber and adapted to receive a flow of the fluid to be treated; and
an output port formed at another part of the flexible flow chamber adapted to receive the flow of the fluid having been treated with the light treatment.

29. The chamber of claim 28 further comprising a flexible body portion, the flow chamber formed within the flexible body portion.

30. The chamber of claim 29 wherein the flexible body portion is substantially flat.

31. The chamber of claim 30 wherein the flexible body portion and the flexible flow chamber comprise two or more sheets of light transmissive material attached together, the flexible flow chamber formed between a portion of the two or more sheets of the light transmissive material.

32. The chamber of claim 28 wherein the flexible flow chamber is comprised of a light transmissive plastic material.

33. The chamber of claim 28 wherein the flexible flow chamber is comprised of a light transmissive material selected from a group consisting of: a polyolefin, a fluorinated polymer, a halogenated polymer, a nylon, and combinations thereof.

34. The chamber of claim 28 wherein the flexible flow chamber is comprised of a light transmissive material selected from a group consisting of: FEP (flourinated ethylene-propylene perfluoro (ethylene-propylene)), EVA (ethylene vinyl acetate), PTFE (polytetrafluoroethylene), PFA (perfluoro (alkoxy alkane)), ethyl vinyl alcohol, polyvinylidene fluoride (PVDF), polyvinyllidine chloride (PVDC): Saran, and polyamides, such as nylon and polychlorotrifluoroethylene (PCTFE): Aclar.

35. The chamber of claim 28 wherein the flow chamber is substantially flat without the fluid flowed therethrough and wherein the fluid expands the flow chamber as the fluid flows therethrough.

36. The chamber of claim 28 wherein the flexible flow chamber is adapted to be restrained between two support structures on either side of the flexible flow chamber.

37. The chamber of claim 28 wherein the disposable treatment chamber is designed to be used once and discarded.

38. The chamber of claim 28 wherein the input port has a substantially circular cross sectional profile and the flexible flow chamber has a substantially flat cross sectional profile, the chamber further comprising:
a taper section formed between the input port and the flexible flow chamber, wherein the taper section is shaped to transition a substantially circular fluid flow profile into a substantially flat fluid flow profile such that the fluid flow through the flexible flow chamber is substantially laminar having approximately the same fluid flow velocity across the profile of the flexible flow chamber.

39. A light treatment device to be illuminated with light for deactivating pathogens within fluid products comprising:
a cartridge body comprising a first part and a second part;
a first light transmissive window of the first part;
a flexible treatment chamber positioned against the first light transmissive window, wherein at least a portion of the flexible treatment chamber is transmissive to at least 1% of the light having at least one wavelength within a range of 170 to 2600 nm, wherein fluid to be treated with the light flows through the flexible treatment chamber; and
a plate portion of the second part, the plate portion positioned against the flexible treatment chamber, wherein the plate portion restrains the flexible treatment chamber against the first light transmissive window in order to define at least one dimensional boundary of a fluid flow path for the fluid within the flexible treatment chamber.

40. The device of claim 39 wherein the flexible treatment chamber comprises:
a flexible body portion;
an input port formed in the flexible body portion;
an output port formed in the flexible body portion; and
a flexible flow chamber formed within the flexible body portion and coupled to the input port and the output port, wherein the flexible flow chamber is transmissive to light, wherein fluid to be treated with light flows through the input port, the flexible flow chamber and the output port, the flexible flow chamber being restrained between the first light transmissive window and the plate portion.

41. The device of claim 39 wherein the plate portion comprises a second light transmissive window.

42. The device of claim 39 wherein the first light transmissive window comprises a first flat light transmissive window and the plate portion comprises a flat plate portion.

43. The device of claim 39 further comprising at least one spacer positioned between the first part and the second part such that the plate portion and the first light transmissive window are separated by a specified distance.

44. The device of claim 39 wherein the flexible treatment chamber includes at least one alignment feature.

45. The device of claim 44 wherein a respective one of the at least one alignment feature is coupled to a respective corresponding alignment feature coupled to one or more of the first part and the second part.

46. A method of fluid decontamination comprising:
flowing a fluid product through a flexible treatment chamber, the flexible treatment chamber being light transmissive to at least 1% of a light treatment having at least one wavelength within a range of 170 to 2600 nm;
illuminating the fluid product with the light as the fluid product is flowed through the flexible treatment chamber; and
deactivating pathogens within the fluid product.

47. The method of claim 46 wherein the fluid product comprises a biological fluid including at least one protein.

48. The method of claim 47 wherein the illuminating step results in a protein damage to kill ratio of less than 5, wherein the protein damage to kill ratio is defined as a percentage of protein activity reduction after the illuminating step divided by the log reduction of the pathogens.

49. The method of claim 46 wherein the illuminating step comprises illuminating the fluid product with pulses of light.

50. The method of claim 49 wherein the illuminating step comprises illuminating the fluid product with the pulses of light having wavelengths within a spectral range of at least between 240 nm and 280 nm and having a pulse duration of less than 100 ms.

51. The method of claim 49 wherein the illuminating step comprises illuminating the fluid product with the pulses of light having a fluence greater than 0.001 J/cm².

52. The method of claim 49 wherein the illuminating step comprises illuminating the fluid product with the pulses of light, wherein at least 0.5% of the fluence of the pulses of light is concentrated at wavelengths within a range of 200 nm to 320 nm.

53. The method of claim 46 wherein the flowing step comprises flowing the fluid product through the flexible treatment chamber at a constant flow rate.
